SEQUENCE LISTING (40 sequences)

SEQ ID NO: 1 - DNA, 224 bp, Psammomys obesus
  misc_feature 15: n = A,T,C or G
atggatgtagacttnggtaatttggattatacaaacaactaaacgttttaagcagaatgagtaatggatcataataatagaatcatggtgctgagggtgatttgaactgtgggaccctgtctcaagaggtttcagggagaagaatattagtatgagacctagggactgttgtgatagtttggtgaagaatgtgactgttttctgcccttgtccaaaaaaaaaaa

SEQ ID NO: 2 - DNA, 128 bp, Psammomys obesus
gacgtagagccgagcgccgagctctcaacaccccagcctccctcagccatttatttattcctgtcccgcctcagcactcagcagtgagcttgaaataaaggcaactttcttgttttcaaaaaaaaaaa

SEQ ID NO: 3 - DNA, 111 bp, Psammomys obesus
tcatttactggtctacatgtctgttttggtggcaatattacattgtttttgtaacagtggttctgtagtgtcctttgaaatcaagtgttcttataactccaaaaaaaaaaa

SEQ ID NO: 4 - DNA, 303 bp, Psammomys obesus
cagaaaaaagtgaaagaaaagctccatgcagttaacgatgaagagtgcactaccctaaaagcaggatggctgtcagaagaatgcatcaatgcaatcatgagcttcgtgtccagaaaagcaaagctgtgaagacccaccacagcagctagacatctcagaggaagaatgtgctgtgagttccagtttgggatacttgaatgacacaaactccactgtgcctttcccttgattaacagagcaatttcgatgagaatgctttacagcactgacaaataaaaactttcataaatctaaaaaaaaaaa

SEQ ID NO: 5 - DNA, 298 bp, Psammomys obesus
  misc_feature 203: n = A,T,C or G
ctgtccatggctggggaaggacctcaccaactgcctgcatctggtcaaggaagagagtgaaaagggggagggtaggagaaggcaccagtggtggcagcaactgcttgttgtgcatgagtctttcccaagggagtcctgaggcccggtccctgttagagggtgggaaatcggaagtggctgctgtggttgaggtgagccctcanaagagctggagcaacccctcccaaggtcccagcactgcttccaaagagcccagcaaaccctgctttcctacacacttgaatggaaaaaaaaaaaa

SEQ ID NO: 6 - DNA, 196 bp, Psammomys obesus
actgagagcttcagtgtttatgttatgaaaatagaaaaagctgaatgcattacacccagagcaaactaggaaggaactaatgaagaataaaaattactgaaattattagaaaacaaaaacaataaaattaaccaaaagctaattctttgaaaatatattaaattgtccctttggcccaattgatgaaaaaaaaaaa

SEQ ID NO: 7 - DNA, 315 bp, Psammomys obesus
  misc_feature 14, 57, 205: n = A,T,C or G
ttctagatagcctnacagctttgctctcatattgtatttaattgctgatacagtatntccttggaggtcttttctctgtaatctacacctctagaattgtttctggcctctgcccatttctgttaacacacagaactctttgggttaccactgcacaaaattgcttatttaggccaggaaatgtcatgaatgtcttccatctcancattatagaggcctaggaggcagaagaaaaagaccaagtttggacaagacaaggctatataaaactggcctcaaaaataaataaaatttcttatctgtgaaaaaaaaaaa

SEQ ID NO: 8 - DNA, 162 bp, Psammomys obesus
  misc_feature 88: n = A,T,C or G
aaaacctggatgtaataaataagatcatggaaagctttatgtgaagaaaattgaatgttatagtataaaaaagatatttatgtatgtncagtttgctaaagccaagttttgtttgttgatttctttgcatttattatagattctataaagtaaaaaaaaaaa

SEQ ID NO: 9 - DNA, 22 bp, Artificial Sequence
  Beta-actin forward primer
gcaaagacctgtatgccaacac

SEQ ID NO: 10 - DNA, 23 bp, Artificial Sequence
  Beta-actin reverse primer
gccagagcagtgatctctttctg

SEQ ID NO: 11 - DNA, 25 bp, Artificial Sequence
  Beta-actin probe
tccggtccacaatgcctgggaacat

SEQ ID NO: 12 - DNA, 24 bp, Artificial Sequence
  AGT-107 forward primer
gcagctagacatctcagaggaaga

SEQ ID NO: 13 - DNA, 21 bp, Artificial Sequence
  AGT-107 reverse primer
ggaaaggcacagtggagtttg

SEQ ID NO: 14 - DNA, 18 bp, Artificial Sequence
  AGT-114 forward primer
caccagtggtggcagcaa

SEQ ID NO: 15 - DNA, 20 bp, Artificial Sequence
  AGT-114 reverse primer
cagcagccacttccgatttc

SEQ ID NO: 16 - DNA, 23 bp, Artificial Sequence
  AGT-116 forward primer
tgaaaatagaaaaagctgaatgc

SEQ ID NO: 17 - DNA, 23 bp, Artificial Sequence
  AGT-116 reverse primer
ttcttcattagttccttcctagc

SEQ ID NO: 18 - DNA, 19 bp, Artificial Sequence
  AGT-115 forward primer
ggcctctgcccatttctgt

SEQ ID NO: 19 - DNA, 23 bp, Artificial Sequence
  AGT-115 reverse primer
attcatgacatttcctggcctaa

SEQ ID NO: 20 - DNA, 29 bp, Artificial Sequence
  AGT-108 forward primer
tggatgtaataaataagatcatggaaagc

SEQ ID NO: 21 - DNA, 25 bp, Artificial Sequence
  AGT-108 reverse primer
aagaaatcaacaaacaaaacttggc

SEQ ID NO: 22 - DNA, 20 bp, Artificial Sequence
  AGT-117 forward primer
tggtgctgagggtgatttga

SEQ ID NO: 23 - DNA, 26 bp, Artificial Sequence
  AGT-117 reverse primer
tatcacaacagtccctaggtctcata

SEQ ID NO: 24 - DNA, 18 bp, Artificial Sequence
  AGT-110 forward primer
gagcgccgagctctcaac

SEQ ID NO: 25 - DNA, 20 bp, Artificial Sequence
  AGT-110 reverse primer
ctgaggcgggacaggaataa

SEQ ID NO: 26 - DNA, 23 bp, Artificial Sequence
  AGT-199 forward primer
acatgtctgttttggtggcaata

SEQ ID NO: 27 - DNA, 26 bp, Artificial Sequence
  AGT-199 reverse primer
tcaaaggacactacagaaccactgtt

SEQ ID NO: 28 - DNA, 337 bp, Psammomys obesus
cacaggacgaaaggcaccatggcactgagcactcagacccaggctgcctgtctcctgctgcttctcattgccagcctgagcagtggtgccattctccagcaacagctcggacagcccgcagcgctccagccgtggcacagggcagaatccagtgccgacaggatgctgatccagacacgaaagaagcgtgacacacacttccccacctgcatattctgctgtcattgctgtaagaatcctggctgcggactgtgctgcaagacgtagagccgagcgccgacctctcaacaccccagcctccctcagccatttatttattcctgtcccgcctcagcct

SEQ ID NO: 29 - protein, 173 aa, Psammomys obesus
  VARIANT 142: Xaa = Any Amino Acid
AISPVMDFCQESETVLENNENKKIEDTEETVLTLSCPDERSERNHVCCLLSISDLTLNEDERASEFAINTGWEGAVHGWGRTSPTACIWSRKRVKRGRVGEGTSGGSNCLLCMSLSQGSPEARSLLEGGKSEVAAVVEVSPXKSWSNPSQGPSTASKEPSKPCFPTHLNGKKK

SEQ ID NO: 30 - protein, 395 aa, Homo sapiens
MDLCQKNETDLENAENNEIQFTEETEPTYTCPDGKSEKNHVYCLLDVSDITLEQDEKAKEFIIGTGWEEAVQGWGRTSPAACIWPRKIPKKARVGEGACSDCLVCVNLSHWSLQTKPPTEGGPEKDQSSPSQTQAAPQGPSTASRAISDICFPTYFRAEKKSLQIKEFIWCNKDWAIPGTNRGKASGNPSGGAHRGLSIPGPLTSRALLVLPPLKALLSNALDVLGKKSKNSFLQSEEKVLDVEKDGCVAYAYGLKTADGKGEKRASELAKHPMVNDTPSSPSPAAQISLLTDPEQRCLHWSLLSEKNLACPPDPSNVRYLAALQLLQKRGVQSYKSKFKAKEPRSPVITRKHVLPKAKQENRPQMLETKVFPRPVLPSLTVSRVIIPVSTHRIL

SEQ ID NO: 31 - protein, 409 aa, Mus musculus
MDVCEESETFLENTENQKIEATEETAPTLHCPDEKSERSHVCCLLGVSDLTLEEDGRASECAISTGWEEAVHGWGRTSPTACIWSKKKVKRGRAREGTNGGNDCLFCMSLSQGSLEPRSLLEVGKLEAGAEAEVSTQKSWSSEKNWSGLSQGPGTASREQSNKLCIPTDVHGEKKSLQLKEFIWCMEEWPMPETVSSKAGRNPSGSPEQGLSTPDSLAAKALVVLPPLKSAPHNLDVLSKKSRNIFWQPEEKVLRVEKDDCMACADGLKGVDGKGEKRHFELASHVKVTNVLPFPPTAAQTHLLSAESQRCCLHWSLLPQKSTVFPPNPSDIHYLATLQVLGQQGKQSCRTRLKTKDTKPPRTTAKHIITEAKQQNRPHVLESKVFPKPLLPSLTVSRVVIPVSTHRVL

SEQ ID NO: 32 - DNA, 248 bp, Homo sapiens
ttaaacagcaagaagatgttaaaaactttaagcaagcatcacagtaatggatctctgtcagaaaaatgagactgacttagaaaatgctgaaaataatgaaattcagttcacagaagaaacagaaccaacctatacttgtccagatggaaaaagtgaaaaaaatcatgtttattgtcttctcgatgtcagtgacattacgcttgaacaagatgaaaaagccaaagagtttattattggaactggatggg

SEQ ID NO: 33 - DNA, 1358 bp, Psammomys obesus
  misc_feature 55, 56, 60, 67, 70, 101, 194, 370, 396, 437, 521, 523,555, 619, 782, 787, 792, 795, 803, 823, 999, 1086, 1130, 1132,1155: n = A,T,C or G
aattcgttatataaaagttaaaaagagaagagaagaatccaggcactgtagcagnngggnaatgttntantttaggtgactgcacactttgtgccaggggngcaaaacacagagctttgttttaatgcaaggagaaggggatgctatcagtacatttatttccagtttgctttcttgccttgtttttcttctgnattccactatacatctaccaagaatataaaggcaccaggactcctgaacactcaggcaatttcccccaattatcaggcagtattaaaaactaaagcagccacagtgagattctactttacactggtgagaatagctatcataacaaatacatcagtttttctttttgttctgatgngtgatgaaagaagccaaaggaaaacnaggctttggcaagaacataaaaaaagttaggaacgttatanattgctaatgcaaatatgaaatatttgtcatttcgtgaaagattggtgttttatcacaaaggttaagtgtggaattgcatgtcncncatagtatatatcaaaaagaaatgaaagctgntcccaaacatttttcacagatgtttgtagcaggagtagtcatcaaagccaaaagctggaaaccncctgagtgtctaccagcagatgattggaataaccaatggtaaatcaatatctaaaacttaactattcagataataaggtctcatatagtccagattggcctagagcatccctcctttgatcttcccaaagaggggattcaggagggagagtgtgactgggcanagagnaggcnggncctatgancaagatgtaaattgtattanttaaaaaaaaagatgaccttgacttctgctactcctgcccctacctactgagtgttggaattacaggcacacacctcatcatgcgcattctttagtgctggtgatcaaacccagggcttcatgcatcctagctaagcactctaccaactcagctatttttcagccctagcaatgtncttctgaatggctcatgggtccaagaggaactcaacagagaaattatagaatagtttacttccatggaaatgaaacctcagcatctnagaattgtgggatattgctgtatcagagggaagtttcaagcttngngtgctccattagcaaacaggaanggaccagactgagagcttcagtgtttatgttatgaaaatagaaaaagctgaatgcattacacccagagcaaactaggaaggaactaatgaagaataaaaattactgaaattattagaaaacaaaaacaataaaattaaccaaaagctaattctttgaaaatatattaaattgtccctttggcccaattgatgaaaaaaaaaaa

SEQ ID NO: 34 - DNA, 947 bp, Psammomys obesus
  misc_feature 144, 371, 588, 808: n = A,T,C or G
cataaaaaaagttaggaacgttatatattgctaatgcaaatatgaaatatttgtcatttcgtgaaagattggtgttttatcacaaaggttaagtgtggaattgcatgtcgcacatagtatatatcaaaaagaaatgaaagctgntcccaaacatttttcacagatgtttgtagcaggagtagtcatcaaagccaaaagctggaaaccacctgagtgtctaccagcagatgattggaataaccaatggtaaatcaatatctaaaacttaactattcagataataaggtctcatatagtccagattggcctagagcatccctcctttgatcttcccaaagaggggattcaggagggagagtgtgactgggcanagaggagggagggcctatgaacaagatgtaaattgaattaattaaaaaaaaagatgaccttgacttctgctactcctgcccctacctactgagtgttggaattacaggcacacacctcatcatgcgcattctttagtgctggtgatcaaacccagggcttcatgcatcctagctaagcactctaccaactcagctatttttcagccctagcaatgtncttctgaatggctcatgggtccaagaggaactcaacagagaaattatagaatagtttacttccatggaaatgaaacctcagcatctcagaattgtgggatattgctgaagcagagggaagtttcaagctttgagtgctccattagcaaagagggagggaccagactgagagcttcagtgtttatgttatgaaaatagaaaaagctgaatgcattacaccnagagcaaactaggaaggaactaatgaagaataaaaattactgaaattattagaaaacaaaaacaataaaattaaccaaaagctaattctttgaaaatatattaaattgtccctttggcccaattgatgaaaaaaaaaaa

SEQ ID NO: 35 - DNA, 554 bp, Psammomys obesus
  misc_feature 459: n = A,T,C or G
tcagggcggggaagaagatgctaaaaactataagcaatcagcccagtaatggatttctgtcaggagagtgaaactgttttagaaaataatgaaaataagaaaattgaagacacagaagaaactgtgctgactttaagttgtccagatgagagaagcgaaaggaatcacgtttgctgtcttctcagtatcagtgatctcacgctgaacgaggatgagcgggccagcgagtttgccatcaacactggatgggagggagctgtccatggctggggaaggacctcaccaactgcctgcatctggtcaaggaagagagtgaaaagggggagggtaggagaaggcaccagtggtggcagcaactgcttgttgtgcatgagtctttcccaagggagtcctgaggcccggtccctgttagagggtgggaaatcggaagtggctgctgtggttgaggtgagccctcanaagagctggagcaacccctcccaaggtcccagcactgcttccaaagagcccagcaaaccctgctttcctacacacttgaatggaaaaaaaaaaaa

SEQ ID NO: 36 - protein, 4 aa, Unknown
  ER membrane retention signal motif in the C-terminus
  VARIANT 1, 2: Xaa = Any Amino Acid
XXRR

SEQ ID NO: 37 - protein, 4 aa, Unknown
  ER membrane retention signal motif in the C-terminus
MCCK

SEQ ID NO: 38 - protein, 4 aa, unknown
  Prenylation motif near the C-terminus
CCKT

SEQ ID NO: 39 - protein, 21 aa, unknown
  sequence fingerprint
  VARIANT 3, 7, 8, 9, 13, 15, 16, 17, 18: Xaa = Any Amino Acid
VSXINGXXXAGGXLXXXXCDY

SEQ ID NO: 40 - protein, 8 aa, UNKNOWN
  conserved (mouse PECI and yeast Eci 1p) motif
  VARIANT 5: Xaa = Val or Ile
  VARIANT 7: Xaa = Ile or Leu
NGPAXGXS

(19) United States
(12) Patent Application Publication
(10) Pub. No.: US 2004/0214188 A1
(43) Pub. Date: Oct. 28, 2004

(54) Gene and uses therefor

(76) Inventors: Greg Collier, Ocean Grove (AU); Ken Walder, Ocean Grove (AU); Julie Elissa Miller, Highton (AU)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/478,443

(22) PCT Filed: May 21, 2002
(86) PCT No.: PCT/AU02/00628

(30) Foreign Application Priority Data
May 21, 2001 (AU) PR 5137

(51) Int. Cl. C12Q 1/68; C07H 21/04; C12N 9/20

(52) U.S. Cl. 435/6; 435/69.1; 435/198; 435/320.1; 435/325; 536/23.2

(57) ABSTRACT

The present invention relates generally to nucleic acid molecules at least expressed in liver or stomach tissue and identified using a differential display or macroarray technique or another technique capable of detecting differential expression of nucleic acid molecules under differing physiological conditions. Expression products from the nucleic acid molecules of the present invention are associated with or act as markers for one or more of a healthy state, obesity, anorexia, weight maintenance, impaired muscle development, diabetes and/or metabolic energy levels and/or altered physiological conditions. The identification of the present nucleic acid molecules and their expression products and/or their derivatives, homologs, analogs and mimetics are proposed to be useful as therapeutic and diagnostic agents or as targets for agents which act as modulators and/or monitors of physiological processes associated with obesity, anorexia, weight maintenance, impaired muscle development, diabetes and/or metabolic energy levels and/or other physiological conditions.

### FIELD OF THE INVENTION

The present invention relates generally to nucleic acid molecules at least expressed in liver or stomach tissue and identified using a differential display or macroarray technique or another technique capable of detecting differential expression of nucleic acid molecules under differing physiological conditions. Expression products from the nucleic acid molecules of the present invention are associated with or act as markers for one or more of a healthy state, obesity, anorexia, weight maintenance, impaired muscle development, diabetes and/or metabolic energy levels and/or altered physiological conditions. The identification of the present nucleic acid molecules and their expression products and/or their derivatives, homologs, analogs and mimetics are proposed to be useful as therapeutic and diagnostic agents or as targets for agents which act as modulators and/or monitors of physiological processes associated with obesity, anorexia, weight maintenance, impaired muscle development, diabetes and/or metabolic energy levels and/or other physiological conditions.

### BACKGROUND OF THE INVENTION

Reference to any prior art in this specification is not, and should not be taken as, an acknowledgment or any form of suggestion that this prior art forms part of the common general knowledge in any country.

The increasing sophistication of recombinant DNA technology is greatly facilitating research and development in the veterinary and allied human and animal health fields. This is particularly the case in the investigation of the genetic bases involved in the etiology of certain disease conditions. One particularly significant condition from the stand point of morbidity and mortality is obesity and its association with type 2 diabetes (formerly non-insulin-dependent diabetes mellitus or NIDDM) and cardiovascular disease.

Obesity is defined as a pathological excess of body fat and is the result of an imbalance between energy intake and energy expenditure for a sustained period of time. Obesity is the most common metabolic disease found in affluent nations. The prevalence of obesity in these nations is alarmingly high, ranging from 10% to upwards of 50% in some subpopulations (Bouchard, The genetics of obesity. Boca Raton: CRC Press, 1994). Of particular concern is the fact that the prevalence of obesity appears to be rising consistently in affluent societies and is now increasing rapidly in less prosperous nations as they become more affluent and/or adopt cultural practices from the more affluent countries (Zimmet, Diabetes Care 15(2): 232-247, 1992).

In 1995 in Australia, for example, 19% of the adult population were obese (BMI>30). On average, women in 1995 weighed 4.8 kg more than their counterparts in 1980 while men weighed 3.6 kg more (Australian Institute of Health and Welfare (AIHW), Heart, Stroke and Vascular diseases, Australian facts. AIHW Cat. No. CVD 7 Canberra: AIHW and the Heart Foundation of Australia, 1999.). More recently, the AusDiab Study conducted between the years 1999 and 2000 showed that 65% of males and 45% of females aged 25-64 years were considered overweight (de Looper and Bhatia, Australia's Health Trends 2001. Australian Institute of Health and Welfare (AIHW) Cat. No. PHE 24. Canberra: AIHW, 2001). The prevalence of obesity in the US also increased substantially between 1991 and 1998, rising from 12% to 18% in Americans during this period (Mokdad et al., JAMA. 282(16): 1519-22, 1999).

The high and increasing prevalence of obesity has serious health implications for both individuals and society as a whole. Obesity is a complex and heterogeneous disorder and has been identified as a key risk indicator of preventable morbidity and mortality since obesity increases the risk of a number of other metabolic conditions including type 2 diabetes mellitus and cardiovascular disease (Must et al., JAMA. 282(16): 1523-1529, 1999; Kopelman, Nature 404: 635-643, 2000). Alongside obesity, the prevalence of diabetes continues to increase rapidly. It has been estimated that there were about 700,000 persons with diabetes in Australia in 1995 while in the US, diabetes prevalence increased from 4.9% in 1990 to 6.9% in 1999 (Mokdad, Diabetes Care 24(2): 412, 2001). In Australia, the annual costs of obesity associated with diabetes and other disease conditions has been conservatively estimated to be AU$810 million for 1992-3 (National Health and Medical Research Council, Acting on Australia's weight: A strategy for the prevention of overweight and obesity. Canberra: National Health and Medical Research Council, 1996). In the US, the National Health Interview Survey (NHIS) estimated the economic cost of obesity in 1995 as approximately US$99 billion, thereby representing 5.7% of total health costs in the U.S. at that time (Wolf and Colditz, Obes Res. 6: 97-106, 1998).

A genetic basis for the etiology of obesity is indicated inter alia from studies in twins, adoption studies and population-based analyses which suggest that genetic effects account for 25-80% of the variation in body weight in the general population (Bouchard [1994; supra]; Kopelman et al., Int J Obesity 18: 188-191, 1994; Ravussin, Metabolism 44(Suppl 3): 12-14, 1995). It is considered that genes determine the possible range of body weight in an individual and then the environment influences the point within this range where the individual is located at any given time (Bouchard [1994; supra]). However, despite numerous studies into genes thought to be involved in the pathogenesis of obesity, there have been surprisingly few significant findings in this area. In addition, genome-wide scans in various population groups have not produced definitive evidence of the chromosomal regions having a major effect on obesity.

A number of organs/tissues have been implicated in the pathophysiology of obesity and type 2 diabetes, and of particular interest is the hypothalamus. The hypothalamus has long been recognized as a key brain area in the regulation of energy intake (Stellar, Psychol Rev 61: 5-22, 1954) and it is now widely accepted that the hypothalamus plays a central role in energy homeostasis, integrating and co-ordinating a large number of factors produced by and/or acting on the hypothalamus.

The stomach is also an important organ. The role of the stomach in regulating food intake is thought to involve two types of signals: the degree of distension of the stomach and the activation of chemoreceptors in the gastric or intestinal wall (Koopmans, Experimental studies on the control of food intake. In: Handbook of Obesity, Ed., G A Bray, C Bouchard, W P T James, pp 273-312, 1998). The gut is the largest endocrine organ in the body and after a meal a large number of gastrointestinal hormones are released. Some examples are gastrin, somatostatin, cholecystokinin, gastric inhibitory polypeptide and neurotensin. Despite general agreement that the stomach provides part of the signal that restricts food intake during a single meal, the nature of this signal or how it is transmitted to the brain remains to be determined. Most likely the information relating to the degree of distension of the stomach or the presence of nutrients in the gastrointestinal walls is transmitted to the brain through either nerves or hormones. The role of the gut hormones identified to date in the regulation of food intake remains to be equivocally determined.

The liver also plays a significant role in a number of important physiological pathways. It has a major role in the regulation of metabolism of glucose, amino acids and fat. In addition the liver is the only organ (other than the gut) that comes into direct contact with a large volume of ingested food and therefore the liver is able to “sense” or monitor the level of nutrients entering the body, particularly the amounts of protein and carbohydrate. It has been proposed that the liver may also have a role in the regulation of food intake through the transmission of unidentified signals relaying information to the brain about nutrient absorption from the gut and metabolic changes throughout the body (Russek, Nature 200: 176, 1963; Koopmans, 1998, supra). The liver also plays a crucial role in maintaining circulating glucose concentrations by regulating pathways such as gluconeogenesis and glycogenolysis. Alterations in glucose homeostasis are important factors in the pathophysiology of impaired glucose tolerance and the development of type 2 diabetes mellitus.

In accordance with the present invention, the subject inventors sought to identify genetic sequences which are differentially expressed in lean and obese animals or in fed compared to unfed animals. Using techniques such as differential display analysis, the inventors identified genes which are proposed to be associated with one or more biological functions connected with a healthy state or a disease condition such as but not limited to obesity, anorexia, weight maintenance, diabetes, muscle development and/or metabolic energy levels and/or other altered physiological conditions. The genetic sequences are in effect molecular markers, the expression of which, provides an indication of the general health status of a subject and can act as targets for therapeutic and diagnostic applications.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A sequence listing is provided after the claims.

Techniques including differential display analysis analysis of genetic material from liver or stomach tissue were used to identify candidate genetic sequences associated with a healthy state or with physiological conditions such as obesity, anorexia, weight maintenance, diabetes, muscle development and/or metabolic energy levels. An animal model was employed comprising the Israeli Sand Rat (Psammomys obesus). Three groups of animals are used designated Groups A, B and C based on metabolic phenotype as follows:—

Group A: lean animals;

Group B: obese, non-diabetic animals; and

Group C: obese, diabetic animals.

Animals were maintained under fed or unfed conditions or under conditions of high or low glucose or insulin and genetic sequences analyzed by differential display techniques. In a preferred embodiment using these techniques, putatively differentially expressed sequences were identified from liver cells designated herein AGT-117, AGT-110 and AGT-199 with sequence identifiers SEQ ID NO:1, SEQ ID NO:2 and SEQ ID NO:3 respectively. Other genetic sequences were identified in stomach tissue as follows: AGT-107, AGT-114, AGT-116, AGT-115 and AGT-108 with sequence identifiers SEQ ID NO:4, SEQ ID NO:5, SEQ ID NO:6, SEQ ID NO:7 and SEQ ID NO:8, respectively.

Differential expression means an elevation in levels of expression of a genetic sequence under one set of conditions compared to another. In one particular embodiment, AGT-117 expression was decreased in pooled animals under fasting conditions. In lean animals, AGT-117 was found to be expressed at a higher level compared to obese, diabetic animals in the fed state. AGT-117 expression correlated negatively with log plasma insulin levels. AGT-110 is expressed at lower levels in pooled animals under fasting conditions. AGT-199 expression was reduced under fasting conditions in lean and obese-diabetic animals. Pooled results indicated that AGT-199 was expressed at lower levels under fasting conditions. AGT-107 was expressed at higher levels under fasting conditions compared to fed conditions. Expression of this gene was negatively correlated with plasma insulin levels. AGT-114 expression was higher in fed animals and expression was negatively correlated with stomach weight. AGT-116 expression was increased in fed animals and was connected to insulin levels. AGT-115 gene expression was higher in fed animals whereas AGT-108 was higher in fasted animals compared to fed animals. A summary of the AGT sequences is provided in Table 1.

The identification of these variably expressed sequences permits the rationale design and/or selection of molecules capable of antagonizing or agonizing the expression products and/or permits the development of screening assays. The screening assays, for example, include assessing the physiological status of a particular subject.

Accordingly, one aspect of the present invention provides a nucleic acid molecule comprising a sequence of nucleotides encoding or complementary to a sequence encoding a protein or a derivative, homolog, analog or mimetic thereof wherein the nucleic acid molecule is expressed in larger or smaller amounts in liver or stomach tissue of obese animals compared to lean animals. Alternatively, or in addition, the nucleic acid molecule is expressed in larger or smaller amounts in liver or stomach tissue of fed animals compared to fasted animals. A fasted animal or fed animal which is fed again is referred to as a “re-fed” animal.

In a preferred embodiment, the nucleic acid molecule comprises a nucleotide sequence substantially as set forth in SEQ ID NO:1 or SEQ ID NO:2 or SEQ ID NO:3 or SEQ ID NO:4 or SEQ ID NO:5 or SEQ ID NO:6 or SEQ ID NO:7 or SEQ BD NO:8 or SEQ ID NO:28 or SEQ ID NO:32 or SEQ ID NO:33 or SEQ ID NO:35 or a nucleotide sequence having at least about 30% similarity to all or part of SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:28 or SEQ ID NO:32 or SEQ ID NO:33 or SEQ ID NO:35 and/or is capable of hybridizing to one or more of SEQ ID NO: 1 or SEQ ID NO:2 or SEQ ID NO:3 or SEQ ID NO:4 or SEQ ID NO:5 or SEQ ID NO:7 or SEQ ID NO:8 or SEQ ID NO:28 or SEQ ID NO:32 or SEQ ID NO:33 or SEQ ID NO:35 or their complementary forms under low stringency conditions at 42° C.

Another aspect of the present invention provides an isolated molecule or a derivative, homolog, analog or mimetic thereof which is produced in a larger or smaller amount in liver or stomach tissue of obese animals compared to lean animals and/or which is produced in a larger or smaller amount in liver or stomach tissue of fed animals compared to fasted animals.

The molecule is generally a protein but may also be an mRNA, intron or exon.

The molecule is encoded by a nucleotide sequence substantially as set forth in SEQ ID NO:1, SEQ ID NO:2, SEQ ID NO:3, SEQ ID NO:4 or SEQ ID NO:5 or SEQ ID NO:6 or SEQ ID NO:7 or SEQ ID NO:8 or SEQ ID NO:28 or SEQ ID NO:32 or SEQ ID NO:33 or SEQ ID NO:35 or a nucleotide sequence having at least 30% similarity to all or part of SEQ ID NO:1, SEQ ID NO:2, SEQ ID NO:3, SEQ ID NO:4 or SEQ ID NO:5 or SEQ ID NO:6 or SEQ ID NO:7 or SEQ ID NO:8 or SEQ ID NO:28 or SEQ ID NO:32 or SEQ ID NO:33 or SEQ ID NO:35 and/or is capable of hybridizing to SEQ ID NO:1, SEQ ID NO:2, SEQ ID NO:3, SEQ ID NO:4 or SEQ ID NO:5 SEQ ID NO:6 or SEQ ID NO:7 or SEQ ID NO:8 or SEQ ID NO:28 or SEQ ID NO:32 or SEQ ID NO:33 or SEQ ID NO:35 under low stringency conditions at 42° C.

In this respect, the molecule may be considered an expression product of the subject nucleotide sequences.

The preferred genetic sequences of the present invention are referred to herein as AGT-117, AGT-110, AGT-199, AGT-107, AGT-114, AGT-116, AGT-115 and AGT-108. The expression product encoded by AGT-117, AGT-110, AGT-199, AGT-107, AGT-114, AGT-116, AGT-115 and AGT-108 are referred to herein as AGT-117, AGT-110, AGT-199, AGT-107, AGT-114, AGT-116, AGT-115 and AGT-108, respectively. The preferred expression products are proteins.

A further aspect of the present invention relates to a composition comprising an expression product such as a protein defined by AGT-117, AGT-110, AGT-199, AGT-107, AGT-114, AGT-116, AGT-115 and AGT-108 or its derivatives, homologs, analogs or mimetics or agonists or antagonists of AGT-117, AGT-110, AGT-199, AGT-107, AGT-114, AGT-116, AGT-115 and AGT-108 together with one or more pharmaceutically acceptable, carriers and/or diluents.

Furthermore, the present invention contemplates a method for treating a subject comprising administering to the subject, a treatment effective amount of AGT-117, AGT-110, AGT-199, AGT-107, AGT-114, AGT-116, AGT-115 and AGT-108 or a derivative, homolog, analog or mimetic thereof or a genetic sequence encoding same or an agonist or antagonist of AGT-117, AGT-110, AGT-199, AGT-107, AGT-114, AGT-116, AGT-115 and AGT-108 or AGT-117, AGT-110, AGT-199, AGT-107, AGT-114, AGT-116, AGT-115 and AGT-108 gene expression for a time and under conditions sufficient to effect treatment.

In accordance with this and other aspects of the present invention, treatments contemplated herein include but are not limited to obesity, anorexia, weight maintenance, energy imbalance and diabetes. Treatment may be by the administration of a pharmaceutical composition br genetic sequences via gene therapy. Treatment is contemplated for human subjects as well as animals such as animals important to livestock industry.

Still another aspect of the present invention is directed to a diagnostic agent for use in monitoring or diagnosing conditions such as but not limited to obesity, anorexia, weight maintenance, energy imbalance and/or diabetes, said diagnostic agent selected from an antibody to AGT-117, AGT-110, AGT-199, AGT-107, AGT-114, AGT-116, AGT-115 and AGT-108 or its derivatives, homologs, analogs or mimetics and a genetic sequence useful in PCR, hybridization, RFLP amongst other techniques.

A summary of sequence identifiers used throughout the subject specification is provided in Table 2.

TABLE 1
Summary of Differentially Expressed Genes

| Previous | | SEQ ID | | | |
|---|---|---|---|---|---|
| Designation* | Gene | NO: | Tissue | Phenotype | Method of Detection |
| L25 | AGT-117 | 1 | Liver | Higher expression | Positively |
| | | | | in fasted compared | correlated with |
| | | | | to fed Sand rats | log plasma |
| | | | | (Groups A, B and | insulin levels |
| | | | | C) | |
| L27 | AGT-110 | 2 | Liver | Lower expression | Positively |
| | | | | in fasted Groups A, | correlated with |
| | | | | B and C | log plasma |
| | | | | | insulin levels |
| L28 | AGT-199 | 3 | Liver | Lower expression | Positively |
| | | | | in fasted Groups A, | correlated with |
| | | | | B and C | log plasma |
| | | | | | insulin levels |
| S6 | AGT-107 | 4 | Stomach | Higher expression | Negatively |
| | | | | in fasted compared | correlated with |
| | | | | to fed animals in | log plasma |
| | | | | Groups A, B | insulin levels |
| S9 | AGT-114 | 5 | Stomach | Higher expression | Negatively |
| | | | | in re-fed compared | correlated with |
| | | | | to fasted animals in | stomach |
| | | | | Groups A, B and C | weight |
| S10 | AGT-116 | 6 | Stomach | Trend for increased | Positively |
| | | | | expression in fed | correlated with |
| | | | | animals in Groups | plasma insulin |
| | | | | A, B and C | levels |
| S15 | AGT-115 | 7 | Stomach | Expression higher | Negatively |
| | | | | in fed animals and | correlated with |
| | | | | re-fed animals in | stomach |
| | | | | Groups A, B and C | weight |
| S31 | AGT-108 | 8 | Stomach | Expression higher | — |
| | | | | in fasted animals | |
| | | | | compared to re-fed | |
| | | | | animals in Groups | |
| | | | | A, B and C | |

*in priority document

A summary of sequence identifiers used throughout the subject specification is provided below.

SUMMARY OF SEQUENCE IDENTIFIERS

| SEQUENCE | |
|---|---|
| ID NO. | DESCRIPTION |
| 1 | partial nucleotide sequence of AGT-117 |
| 2 | partial nucleotide sequence of AGT-110 |
| 3 | partial nucleotide sequence of AGT-199 |
| 4 | partial nucleotide sequence of AGT-107 |
| 5 | partial nucleotide sequence of AGT-114 |
| 6 | partial nucleotide sequence of AGT-116 |
| 7 | partial nucleotide sequence of AGT-115 |
| 8 | partial nucleotide sequence of AGT-108 |
| 9 | β-actin forward primer |
| 10 | β-actin reverse primer |
| 11 | β-actin probe |
| 12 | AGT-107 forward primer |
| 13 | AGT-107 reverse primer |
| 14 | AGT-114 forward primer |
| 15 | AGT-114 reverse primer |
| 16 | AGT-116 forward primer |
| 17 | AGT-116 reverse primer |
| 18 | AGT-115 forward primer |
| 19 | AGT-115 reverse primer |
| 20 | AGT-108 forward primer |
| 21 | AGT-108 reverse primer |
| 22 | AGT-117 forward primer |
| 23 | AGT-117 reverse primer |
| 24 | AGT-110 forward primer |
| 25 | AGT-110 reverse primer |
| 26 | AGT-199 forward primer |
| 27 | AGT-199 reverse primer |
| 28 | Partial nucleotide sequence of AGT-110 |
| 29 | Partial amino acid sequence of AGT-114 |
| 30 | Corresponding human AGT-114 amino acid sequence |
| 31 | Corresponding murine AGT-114 amino acid sequence |
| 32 | Corresponding human AGT-114 genomic nucleotide |
| | sequence |
| 33 | Nucleotide sequence of AGT-116 |
| 34 | Nucleotide sequence of AGT-116 |
| 35 | Nucleotide sequence of AGT-114 |

### BRIEF DESCRIPTION OF THE FIGURES

FIG. 1 is a graphical representation of the expression values of AGT-117 in fed and fasted livers of 3 groups of Israeli sand rats (Group A, B & C, refer to page 18 for details) in either the fed (fed) state or fasted (fast) state.

FIG. 2 is a graphical representation of the mean expression values of AGT-117 in fed or fasted livers of all Israeli sand rats tested. Fed (fed) states or fasted (fast) state.

FIG. 3 is a graphical representation of the LG10 of AGT-117 expression values versus the LG10 of insulin concentration in all Israeli sand Rats tested.

FIG. 4 is a graphical representation of the expression values of AGT-110 in fed and fasted livers of 3 groups of Israeli sand rats (Group A, B & C, refer to page 18 for details) in either the fed (fed) state or fasted (fast) state.

FIG. 5 is a graphical representation of the mean expression values of AGT-110 in fed or fasted livers of all Israeli sand rats tested. Fed (fed) states or fasted (fast) state.

FIG. 6 is a graphical representation of the AGT-110 expression values versus the LG10 of insulin concentration in all Israeli sand rats tested.

FIG. 7 is a graphical representation of the expression values of AGT-199 in fed and fasted livers of 3 groups of Israeli sand rats (Group A, B & C, refer to page 18 for details) in either the fed (fed) state or fasted (fast) state.

FIG. 8 is a graphical representation of the mean expression values of AGT-199 in fed or fasted livers of all Israeli sand rats tested. Fed (fed) states or fasted (fast) state.

FIG. 9 is a graphical representation of AGT-199 expression values versus the LG10 of insulin in all Israeli sand rats tested.

FIG. 10 is a graphical representation of the mean expression values of AGT-107 in fed, fasted or re-fed stomach tissue of Israeli sand rats. Fed (fed) state, fasted (fast) state, Re-fed (re-fed) state; n≧13 for each condition tested.

FIG. 11 is a graphical representation of AGT-107 expression values versus insulin in all Israeli sand rats tested.

FIG. 12 is a graphical representation of the mean expression values of AGT-114 in fed, fasted or re-fed stomach tissue of Israeli sand rats. Fed (fed) state, fasted (fast) state, re-fed (re-fed) state; n≧13 for each condition tested.

FIG. 13 is a graphical representation of AGT-114 expression values versus stomach weight in all Israeli sand rats tested.

FIG. 14 is a graphical representation of the mean expression values of AGT-116 in fed, fasted or re-fed stomach tissue of Israeli sand rats. Fed (fed) state, fasted (fast) state, re-fed (re-fed) state; n≧13 for each condition tested.

FIG. 15 is a graphical representation of AGT-116 expression values versus the LG10 of insulin in all Israeli sand rats tested.

FIG. 16 is a graphical representation of the mean expression values of AGT-115 in fed, fasted or re-fed stomach tissue of Israeli sand rats. Fed (fed) state, fasted (fast) state, re-fed (re-fed) state; n≧13 for each condition tested.

FIG. 17 is a graphical representation LG10 of AGT-115 expression values versus stomach content in all Israeli sand rats tested.

FIG. 18 is a graphical representation LG10 of AGT-115 expression values versus stomach weight in all Israeli sand rats tested.

FIG. 19 is a graphical representation of the mean expression values of AGT-108 in fed, fasted or re-fed stomach tissue of Israeli sand rats. Fed (fed) state, fasted (fast) state, re-red (re-fed) state; n≧13 for each condition tested.

### DETAILED DESCRIPTION OF THE PREFERRED EMBODIMENTS

The present invention is predicated in part on the identification of novel genes associated inter alia with regulation of energy balance obesity and diabetes and/or muscle development. The genes were identified by a number of procedures including differential screening of liver or stomach mRNA between lean and obese animals and/or between fed animals and fasted animals.

The term “differential” array is used in its broadest sense to include the expression of nucleic acid sequences in one type of tissue relative to another type of tissue in the same or different animals. Reference to “different” animals include the same animals but in different gastronomical states such as in a fed or non-fed state.

Accordingly, one aspect of the present invention provides a nucleic acid molecule comprising a sequence of nucleotides encoding or complementary to a sequence encoding an expression product or a derivative, homolog, analog or mimetic thereof wherein said nucleic acid molecule is expressed in larger or smaller amounts in liver or stomach tissue of obese animals compared to lean animals.

In a related embodiment, there is provided a nucleic acid molecule comprising a sequence of nucleotides encoding or complementary to a sequence encoding an expression product or a derivative, homolog, analog or mimetic thereof wherein said nucleic acid molecule is expressed in larger or smaller amounts in liver or stomach tissue of fed animals compared to fasted animals.

The expression product may be a protein or mRNA or may be an exon or intron spliced, for example, from an RNA construct.

The terms “lean” and “obese” are used in their most general sense but should be considered relative to the standard criteria for determining obesity. Generally, for human subjects, the definition of obesity is BMI>30 (Risk Factor Prevalence Study Management Committee. Risk Factor Prevalence Study: Survey No. 3 1989. Canberra: National Heart Foundation of Australia and Australian Institute of Health, 1990; Waters and Bennett, Risk Factors for cardiovascular disease: A summary of Australian data. Canberra: Australian Institute of Health and Welfare, 1995).

Conveniently, an animal model may be employed to study the effects of obese and lean animals. In particular, the present invention is exemplified using the Psammomys obesus (the Israeli sand rat) animal model of dietary-induced obesity and NIDDM. In its natural desert habitat, an active lifestyle and saltbush diet ensure that they remain lean and normoglycemic (Shafrir and Gutman, J Basic Clin Physiol Pharm 4: 83-99, 1993). However, in a laboratory setting on a diet of ad libitum chow (on which many other animal species remain healthy), a range of pathophysiological responses are seen (Barnett et al., Diabetologia 37: 671-676, 1994a; Barnett et al., Int. J. Obesity 18: 789-794, 1994b, Barnett et al., Diabete Nutr Metab 8: 4247, 1995). By the age of 16 weeks, more than half of the animals become obese and approximately one third develop NIDDM. Only hyperphagic animals go on to develop hyperglycemia, highlighting the importance of excessive energy intake in the pathophysiology of obesity and NIDDM in Psammomys obesus (Collier et al., Ann New York Acad Sci 827: 50-63, 1997a; Walder et al., Obesity Res 5: 193-200, 1997a). Other phenotypes found include hyperinsulinemia, dyslipidemia and impaired glucose tolerance (Collier et al., 1997a; supra; Collier et al., Exp Clin Endocrinol Diabetes 105: 36-37, 1997b). Psammomys obesus exhibit a range of bodyweight and blood glucose and insulin levels which forms a continuous curve that closely resembles the patterns found in human populations, including the inverted U-shaped relationship between blood glucose and insulin levels known as “Starling's curve of the pancreas” (Barnett et al., 1994a; supra). It is the heterogeneity of the phenotypic response of Psammomys obesus which make it an ideal model to study the etiology and pathophysiology of obesity and NIDDM.

Psammomys obesus animals are conveniently divided into three groups viz Group A animals which are lean, normoglycemic and normoinsuinemic, Group B animals which are obese, normoglycemic and hyperinuslinemic and Group C animals which are obese, hyperglycemic and hyperinsulinemic.

Another aspect of the present invention provides a nucleic acid molecule comprising a nucleotide sequence encoding or complementary to a sequence encoding an expression product or a derivative, homolog, analog or mimetic thereof wherein said nucleotide sequence is as substantially set forth in SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:28 or SEQ ID NO:32 or SEQ ID NO:33 or SEQ ID NO:35 a nucleotide sequence having at least about 30% similarity to all or part of SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:28 or SEQ ID NO:32 or SEQ ID NO:33 or SEQ ID NO:35 and/or is capable of hybridizing to one or more of SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 OR SEQ ID NO:28 or SEQ ID NO:32 or SEQ ID NO:33 or SEQ ID NO:35 or their complementary forms under low stringency conditions at 42° C. and wherein said nucleic acid molecule is expressed in a larger or smaller amount in liver or stomach tissue of obese animals compared to lean animals and/or in fed animals compared to fasted animals.

Reference herein to similarity is generally at a level of comparison of at least 15 consecutive or substantially consecutive nucleotides or at least 5 consecutive or substantially consecutive amino acid residues. Preferred percentage similarities have at least about 40%, at least about 50%, at least about 60%, at least about 70%, at least about 80% and at least about 90% or above.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (Nucl. Acids Res. 25: 3389, 1997). A detailed discussion of sequence analysis can be found in Unit 19.3 of Ausubel et al. (“Current Protocols in Molecular Biology” John Wiley & Sons Inc, 1994-1998, Chapter 15).

The terms “sequence similarity” and “sequence identity” as used herein refers to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (version 2.5 for windows; available from Hitachi Software engineering Co., Ltd., South San Francisco, Calif., USA) using standard defaults as used in the reference manual accompanying the software. Similar comments apply in relation to sequence similarity.

Reference herein to a low stringency includes and encompasses from at least about 0 to at least about 15% v/v formamide and from at least about 1 M to at least about 2 M salt for hybridization, and at least about 1 M to at least about 2 M salt for washing conditions.

Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C)% (Marmur and Doty, J. Mol. Biol. 5: 109, 1962). However, the Tₘ of a duplex DNA decreases by 1° C. with every increase of 1% in the number of mismatch base pairs (Bonner and Laskey, Eur. J. Biochem. 46: 83, 1974. Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The nucleotide sequence or amino acid sequence of the present invention may correspond to exactly the same sequence of the naturally occurring gene (or corresponding cDNA) or protein or may carry one or more nucleotide or amino acid substitutions, additions and/or deletions. The nucleotide sequences set forth in SEQ ID NO:1, SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:28 or SEQ ID NO:32 or SEQ ID NO:33 or SEQ ID NO:35 correspond to the genes referred to herein as AGT-117, AGT-110, AGT-199, AGT-107, AGT-114, AGT-116, AGT-115 and AGT-108, respectively. The corresponding proteins are AGT-117, AGT-110, AGT-199, AGT-107, AGT-114, AGT-116, AGT-115 and AGT-108, respectively. Reference herein to AGT-117, AGT-110, AGT-199, AGT-107, AGT-114, AGT-116, AGT-115 and AGT-108 includes, where appropriate, reference to the genomic gene or cDNA as well as any naturally occurring or induced derivatives. Apart from the substitutions, deletions and/or additions to the nucleotide sequence, the present invention further encompasses mutants, fragments, parts and portions of the nucleotide sequence corresponding to AGT-117, AGT-110, AGT-199, AGT-107, AGT-114, AGT-116, AGT-115 and AGT-108.

The expression pattern of AGT-117, AGT-110, AGT-199, AGT-107, AGT-114, AGT-116, AGT-115 and AGT-108 has been determined, inter alia, to indicate an involvement in the regulation of one or more obesity, diabetes and/or energy metabolism. In addition to the differential expression of AGT-117, AGT-110, AGT-199, AGT-107, AGT-114, AGT-116, AGT-115 and AGT-108 in liver or stomach tissue of lean versus obese animals and fed versus fasted animals, these genes may also be expressed in other tissues including but in no way limited to hypothalamus, cerebellum or subscapular fat or or adrenal gland. The subject nucleic acid molecules are preferably a sequence of deoxyribonucleic acids such as a cDNA sequence or a genomic sequence. A genomic sequence may also comprise exons and introns. A genomic sequence may also include a promoter region or other regulatory regions. The present invention extends, however, to mRNA, introns and exons which may also be involved in genetic networking, whether or not they are translated into proteins.

A homolog is considered to be a AGT-117, AGT-110, AGT-199, AGT-107, AGT-114, AGT-116, AGT-115 or AGT-108 gene from another animal species. The AGT-117, AGT-110, AGT-199, AGT-107, AGT-114, AGT-116, AGT-115 or AGT-108 gene is exemplified herein from Psammomys obesus liver or stomach. The invention extends, however, to the homologous gene, as determined by nucleotide sequence and/or function, from humans, primates, livestock animals (e.g. cows, sheep, pigs, horses, donkeys), laboratory test animals (e.g. mice, guinea pigs, hamsters, rabbits), companion animals (e.g. cats, dogs) and captured wild animals (e.g. rodents, foxes, deer, kangaroos).

The nucleic acid of the present invention and in particular AGT-117, AGT-110, AGT-199, AGT-107, AGT-114, AGT-116, AGT-115 and AGT-108 and its derivatives and homologs may be in isolated or purified form and/or may be ligated to a vector such as an expression vector. Expression may be in a eukaryotic cell line (e.g. mammalian, insect or yeast cells) or in microbial cells (e.g. E. coli) or both.

The derivatives of the nucleic acid molecule of the present invention include oligonucleotides, PCR primers, antisense molecules, molecules suitable for use in co-suppression and fusion nucleic acid molecules. Ribozymes and DNA enzymes are also contemplated by the present invention directed to AGT-117, AGT-110, AGT-199, AGT-107, AGT-114, AGT-116, AGT-115 and AGT-108 or its mRNA. Derivatives and homologs of AGT-117, AGT-110, AGT-199, AGT-107, AGT-114, AGT-116, AGT-115 and AGT-108 are conveniently encompassed by those nucleotide sequences capable of hybridizing to SEQ ID NO:1, SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:28 or SEQ ID NO:32 or SEQ ID NO:33 or SEQ ID NO:35 under low stringency conditions at 42° C.

The present invention extends to expression products of AGT-117, AGT-110, AGT-199, AGT-107, AGT-114, AGT-116, AGT-115 and AGT-108. The preferred expression products are proteins or mutants, derivatives, homologs or analogs thereof.

Derivatives include fragments, parts, portions, mutants, variants and mimetics from natural, synthetic or recombinant sources including fusion proteins. Parts or fragments include, for example, active regions of AGT-117, AGT-110, AGT-199, AGT-107, AGT-114, AGT-116, AGT-115 or AGT-108. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

Chemical and functional equivalents of AGT-117, AGT-110, AGT-199, AGT-107, AGT-114, AGT-116, AGT-115 or AGT-108 should be understood as molecules exhibiting any one or more of the functional activities of these molecules and may be derived from any source such as being chemically synthesized or identified via screening processes such as natural product screening.

The derivatives include fragments having particular epitopes or parts of the entire protein fused to peptides, polypeptides or other proteinaceous or non-proteinaceous molecules.

Another aspect of the present invention provides an isolated protein or a derivative, homolog, analog or mimetic thereof which is produced in larger amounts in liver or stomach tissue in obese animals compared to lean animals and/or in fed compared to fasted animals.

In a more preferred aspect of the present invention, there is provided an isolated protein or a derivative, homolog, analog or mimetic thereof wherein said protein comprises an amino acid sequence substantially encoded by a nucleotide sequence as set forth in SEQ ID NO:1, SEQ ID NO:2, SEQ ID NO:3, SEQ ID NO:4 or SEQ ID NO:5 or SEQ ID NO:6 or SEQ ID NO:7 or SEQ ID NO:8 or SEQ ID NO:28 or SEQ ID NO:32 or SEQ ID NO:33 or SEQ ID NO:35 or an amino acid sequence having at least 30% similarity to all or part thereof and wherein said protein is produced in a larger or smaller amount in liver or stomach tissue of obese animals compared to lean animals and/or in fed compared to fasted animals. A fed animal in this case includes a re-fed animal.

A further aspect of the present invention is directed to an isolated protein or a derivative, homolog, analog or mimetic thereof wherein said protein is encoded by a nucleotide sequence substantially as set forth in SEQ ID NO:1, SEQ ID NO:2, SEQ ID NO:3, SEQ ID NO:4 or SEQ ID NO:5 or SEQ ID NO:6 or SEQ ID NO:7 or SEQ ID NO:8 or SEQ ID NO:28 or SEQ ID NO:32 or SEQ ID NO:33 or SEQ ID NO:35 or a nucleotide sequence having at least 30% similarity to all or part of SEQ ID NO:1, SEQ ID NO:2, SEQ ID NO:3, SEQ ID NO:4 or SEQ ID NO:5 or SEQ ID NO:6 or SEQ ID NO:7 or SEQ ID NO:8 or SEQ ID NO:28 or SEQ ID NO:32 or SEQ ID NO:33 or SEQ ID NO:35 and/or is capable of hybridizing to SEQ ID NO:1, SEQ ID NO:2, SEQ ID NO:3, SEQ ID NO:4 or SEQ ID NO:5 or SEQ ID NO:6 or SEQ ID NO:7 or SEQ ID NO:8 or SEQ ID NO:28 or SEQ ID NO:32 or SEQ ID NO:33 or SEQ ID NO:35 or their complementary forms under low stringency conditions at 42° C.

Reference herein to AGT-117, AGT-110, AGT-199, AGT-107, AGT-114, AGT-116, AGT-115 and AGT-108 includes reference to isolated or purified naturally occurring AGT-117, AGT-110, AGT-199, AGT-107, AGT-114, AGT-116, AGT-115 and AGT-108 protein molecules as well as any derivatives, homologs, analogs and mimetics thereof. Derivatives include parts, fragments and portions of AGT-117, AGT-110, AGT-199, AGT-107, AGT-114, AGT-116, AGT-115 and AGT-108 as well as single and multiple amino acid substitutions, deletions and/or additions to AGT-117, AGT-110, AGT-199, AGT-107, AGT-114, AGT-116, AGT-115 and AGT-108. A derivative of AGT-117, AGT-110, AGT-199, AGT-107, AGT-114, AGT-116, AGT-115 and AGT-108 is conveniently encompassed by molecules encoded by a nucleotide sequence capable of hybridizing to SEQ ID NO:1, SEQ ID NO:2, SEQ ID NO:3, SEQ ID NO:4 or SEQ ID NO:5 or SEQ ID NO:6 or SEQ ID NO:7 or SEQ ID NO:8 or SEQ ID NO:28 or SEQ ID NO:32 or SEQ ID NO:33 or SEQ ID NO:35 under low stringency conditions at 42° C.

Other derivatives of AGT-117, AGT-110, AGT-199, AGT-107, AGT-114, AGT-116, AGT-115 and AGT-108 include chemical analogs. Analogs of AGT-117, AGT-110, AGT-199, AGT-107, AGT-114, AGT-116, AGT-115 and AGT-108 contemplated herein include, but are not limited to, modifications to side chains, incorporation of unnatural amino acids and/or their derivatives during peptide, polypeptide or protein synthesis and the use of crosslinkers and other methods which impose confirmational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 3.

TABLE 3

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodliimide (COOH). In addition, peptides can be conformationally constrained by, for example, incorporation of C_{α} and N _{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

All such modifications may also be useful in stabilizing the AGT-117, AGT-110, AGT-199, AGT-107, AGT-114, AGT-116, AGT-115 and AGT-108 molecule for use in in vivo administration protocols or for diagnostic purposes.

The nucleic acid molecule of the present invention is preferably in isolated form or ligated to a vector, such as an expression vector. By “isolated” is meant a nucleic acid molecule having undergone at least one purification step and this is conveniently defined, for example, by a composition comprising at least about 10% subject nucleic acid molecule, preferably at least about 20%, more preferably at least about 30%, still more preferably at least about 40-50%, even still more preferably at least about 60-70%, yet even still more preferably 80-90% or greater of subject nucleic acid molecule relative to other components as determined by molecular weight, encoding activity, nucleotide sequence, base composition or other convenient means. The nucleic acid molecule of the present invention may also be considered, in a preferred embodiment, to be biologically pure.

The term “protein” should be understood to encompass peptides, polypeptides and proteins. The protein may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “protein” includes a protein comprising a sequence of amino acids as well as a protein associated with other molecules such as amino acids, lipids, carbohydrates or other peptides, polypeptides or proteins.

In a particularly preferred embodiment, the nucleotide sequence corresponding to AGT-117 is a cDNA sequence comprising a sequence of nucleotides as set forth in SEQ ID NO: 1 or a derivative, homolog or analog thereof including a nucleotide sequence having similarity to SEQ ID NO: 1.

In another particularly preferred embodiment, the nucleotide sequence corresponding to AGT-110 is a cDNA sequence comprising a sequence of nucleotides as set forth in SEQ ID NO:2 or a derivative, homolog or analog thereof including a nucleotide sequence having similarity to SEQ ID NO:2.

In still another particularly preferred embodiment, the nucleotide sequence corresponding to AGT-199 is a cDNA sequence comprising a sequence of nucleotides as set forth in SEQ ID NO:3 or a derivative, homolog or analog thereof including a nucleotide sequence having similarity to SEQ ID NO:3.

In a further particularly preferred embodiment, the nucleotide sequence corresponding to AGT-107 is a cDNA sequence comprising a sequence of nucleotides as set forth in SEQ ID NO:4 or a derivative, homolog or analog thereof including a nucleotide sequence having similarity to SEQ ID NO:4.

In still a further particularly preferred embodiment, the nucleotide sequence corresponding to AGT-114 is a cDNA sequence comprising a sequence of nucleotides as set forth in SEQ ID NO:5 or a derivative, homolog or analog thereof including a nucleotide sequence having similarity to SEQ ID NO:5.

In yet another particularly preferred embodiment, the nucleotide sequence corresponding to AGT-116 is a cDNA sequence comprising a sequence of nucleotides as set forth in SEQ ID NO:5 or a derivative, homolog or analog thereof including a nucleotide sequence having similarity to SEQ ID NO:6.

In still yet another particularly preferred embodiment, the nucleotide sequence corresponding to AGT-115 is a cDNA sequence comprising a sequence of nucleotides as set forth in SEQ ID NO:5 or a derivative, homolog or analog thereof including a nucleotide sequence having similarity to SEQ ID NO:7.

In even still a further particularly preferred embodiment, the nucleotide sequence corresponding to AGT-108 is a cDNA sequence comprising a sequence of nucleotides as set forth in SEQ ID NO:8 or a derivative, homolog or analog thereof including a nucleotide sequence having similarity to SEQ ID NO:8.

In another particularly preferred embodiment, the nucleotide sequence corresponding to AGT-110 is a cDNA sequence comprising a sequence of nucleotides as set forth in SEQ ID NO:28 or a derivative, homolog or analog thereof including a nucleotide sequence having similarity to SEQ ID NO:28.

In still another particularly preferred embodiment, the nucleotide sequence corresponding to AGT-114 is a cDNA sequence comprising a sequence of nucleotides as set forth in SEQ ID NO:32 or a derivative, homolog or analog thereof including a nucleotide sequence having similarity to SEQ ID NO:32.

In a further particularly preferred embodiment, the nucleotide sequence corresponding to AGT-116 is a cDNA sequence comprising a sequence of nucleotides as set forth in SEQ ID NO:33 or a derivative, homolog or analog thereof including a nucleotide sequence having similarity to SEQ ID NO:33.

In another particularly preferred embodiment, the nucleotide sequence corresponding to AGT-114 is a cDNA sequence comprising a sequence of nucleotides as set forth in SEQ ID NO:35 or a derivative, homolog or analog thereof including a nucleotide sequence having similarity to SEQ ID NO:35.

The nucleic acid molecule may be ligated to an expression vector capable of expression in a prokaryotic cell (e.g. E. coli) or a eukaryotic cell (e.g. yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector. The latter embodiment facilitates production of recombinant forms of sphingosine kinase which forms are encompassed by the present invention.

The present invention extends to the expression product of the nucleic acid molecules as hereinbefore defined. The expression product is preferably a protein but extends to mRNA, RNA, introns and exons.

Preferably, the expression products are AGT-117, AGT-110, AGT-199, AGT-107, AGT-114, AGT-116, AGT-115 and AGT-108 encoded by SEQ ID NO:1, SEQ ID NO:2, SEQ ID NO:3, SEQ ID NO:4 or SEQ ID NO:5 or SEQ ID NO:6 or SEQ ID NO:7 or SEQ ID NO:8 or SEQ ID NO:28 or SEQ ID NO:32 or SEQ ID NO:33 or SEQ ID NO:35, respectively or are derivatives, analogs, homologs, chemical equivalents or mimetics thereof.

Another aspect of the present invention is directed to an isolated protein selected from the list consisting of:—

(i) a protein encoded by a nucleic acid molecule which molecule is differentially expressed in liver or stomach tissue of obese animals compared to lean animals or a derivative, homolog, analog, chemical equivalent or mimetic thereof;

(ii) a protein encoded by a nucleic acid molecule which molecule is differentially expressed in liver or stomach tissue of fed animals compared to fasted animals or a derivative, homolog, analog, chemical equivalent or mimetic thereof;

(iii) a protein encoded by a nucleotide sequence substantially as set forth in SEQ ID NO:1 or a derivative, homolog or analog thereof or a sequence encoding an amino acid sequence having at least about 30% similarity to this sequence or a derivative, homolog, analog, chemical equivalent or mimetic of said protein;

(iv) a protein encoded by a nucleotide sequence substantially as set forth in SEQ ID NO:2 or a derivative, homolog or analog thereof or a sequence encoding an amino acid sequence having at least about 30% similarity to this sequence or a derivative, homolog, analog, chemical equivalent or mimetic of said protein;

(v) a protein encoded by a nucleotide sequence substantially as set forth in SEQ ID NO:3 or a derivative, homolog or analog thereof or a sequence encoding an amino acid sequence having at least about 30% similarity to this sequence or a derivative, homolog, analog, chemical equivalent or mimetic of said protein;

(vi) a protein encoded by a nucleotide sequence substantially as set forth in SEQ ID NO:4 or a derivative, homolog or analog thereof or a sequence encoding an amino acid sequence having at least about 30% similarity to this sequence or a derivative, homolog, analog, chemical equivalent or mimetic of said protein;

(vii) a protein encoded by a nucleotide sequence substantially as set forth in SEQ ID NO:5 or a derivative, homolog or analog thereof or a sequence encoding an amino acid sequence having at least about 30% similarity to this sequence or a derivative, homolog, analog, chemical equivalent or mimetic of said protein;

(viii) a protein encoded by a nucleotide sequence substantially as set forth in SEQ ID NO:6 or a derivative, homolog or analog thereof or a sequence encoding an amino acid sequence having at least about 30% similarity to this sequence or a derivative, homolog, analog, chemical equivalent or mimetic of said protein;

(ix) a protein encoded by a nucleotide sequence substantially as set forth in SEQ ID NO:7 or a derivative, homolog or analog thereof or a sequence encoding an amino acid sequence having at least about 30% similarity to this sequence or a derivative, homolog, analog, chemical equivalent or mimetic of said protein;

(x) a protein encoded by a nucleotide sequence substantially as set forth in SEQ ID NO:8 or a derivative, homolog or analog thereof or a sequence encoding an amino acid sequence having at least about 30% similarity to this sequence or a derivative, homolog, analog, chemical equivalent or mimetic of said protein;

(xi) a protein encoded by a nucleotide sequence substantially as set forth in SEQ ID NO:28 or a derivative, homolog or analog thereof or a sequence encoding an amino acid sequence having at least about 30% similarity to this sequence or a derivative, homolog, analog, chemical equivalent or mimetic of said protein;

(xii) a protein encoded by a nucleotide sequence substantially as set forth in SEQ ID NO:32 or a derivative, homolog or analog thereof or a sequence encoding an amino acid sequence having at least about 30% similarity to this sequence or a derivative, homolog, analog, chemical equivalent or mimetic of said protein;

(xiii) a protein encoded by a nucleotide sequence substantially as set forth in SEQ ID NO:33 or a derivative, homolog or analog thereof or a sequence encoding an amino acid sequence having at least about 30% similarity to this sequence or a derivative, homolog, analog, chemical equivalent or mimetic of said protein;

(xiv) a protein encoded by a nucleotide sequence substantially as set forth in SEQ ID NO:35 or a derivative, homolog or analog thereof or a sequence encoding an amino acid sequence having at least about 30% similarity to this sequence or a derivative, homolog, analog, chemical equivalent or mimetic of said protein;

(xv) a protein encoded by a nucleic acid molecule capable of hybridizing to the nucleotide sequence as set forth in SEQ ID NO:1 or a derivative, homolog or analog thereof under low stringency conditions;

(xvi) a protein encoded by a nucleic acid molecule capable of hybridizing to the nucleotide sequence as set forth in SEQ ID NO:2 or a derivative, homolog or analog thereof under low stringency conditions;

(xvii) a protein encoded by a nucleic acid molecule capable of hybridizing to the nucleotide sequence as set forth in SEQ ID NO:3 or a derivative, homolog or analog thereof under low stringency conditions;

(xviii) a protein encoded by a nucleic acid molecule capable of hybridizing to the nucleotide sequence as set forth in SEQ ID NO:4 or a derivative, homolog or analog thereof under low stringency conditions;

(xix) a protein encoded by a nucleic acid molecule capable of hybridizing to the nucleotide sequence as set forth in SEQ ID NO:5 or a derivative, homolog or analog thereof under low stringency conditions;

(xx) a protein encoded by a nucleic acid molecule capable of hybridizing to the nucleotide sequence as set forth in SEQ ID NO:6 or a derivative, homolog or analog thereof under low stringency conditions;

(xxi) a protein encoded by a nucleic acid molecule capable of hybridizing to the nucleotide sequence as set forth in SEQ ID NO:7 or a derivative, homolog or analog thereof under low stringency conditions;

(xxii) a protein encoded by a nucleic acid molecule capable of hybridizing to the nucleotide sequence as set forth in SEQ ID NO:8 or a derivative, homolog or analog thereof under low stringency conditions;

(xxiii) a protein encoded by a nucleic acid molecule capable of hybridizing to the nucleotide sequence as set forth in SEQ ID NO:28 or a derivative, homolog or analog thereof under low stringency conditions;

(xxiv) a protein encoded by a nucleic acid molecule capable of hybridizing to the nucleotide sequence as set forth in SEQ ID NO:32 or a derivative, homolog or analog thereof under low stringency conditions;

(xxv) a protein encoded by a nucleic acid molecule capable of hybridizing to the nucleotide sequence as set forth in SEQ ID NO:33 or a derivative, homolog or analog thereof under low stringency conditions;

(xxvi) a protein encoded by a nucleic acid molecule capable of hybridizing to the nucleotide sequence as set forth in SEQ ID NO:35 or a derivative, homolog or analog thereof under low stringency conditions;

(xxvii) a protein as defined in any one of paragraphs (i) to (xxvi) in a homodimeric form; and

(xxviii) a protein as defined in any one of paragraphs (i) to (xxvi) in a heterodimeric form.

The protein of the present invention is preferably in isolated form. By “isolated” is meant a protein having undergone at least one purification step and this is conveniently defined, for example, by a composition comprising at least about 10% subject protein, preferably at least about 20%, more preferably at least about 30%, still more preferably at least about 40-50%, even still more preferably at least about 60-70%, yet even still more preferably 80-90% or greater of subject protein relative to other components as determined by molecular weight, amino acid sequence or other convenient means. The protein of the present invention may also be considered, in a preferred embodiment, to be biologically pure.

Without limiting the theory or mode of action of the present invention, the expression of AGT-117, AGT-110, AGT-199, AGT-107, AGT-114, AGT-116, AGT-115 and/or AGT-108 is thought to relate to body weight and circulating triglycerides. Modulation of these genes expression is thought, inter alia, to regulate energy balance via effects on energy intake and also effects on carbohydrate/fat metabolism. The expression of these genes may also be regulated by fasting and feeding, accordingly, regulating the expression and/or activity of these genes or their expression products could provide a mechanism for regulating both body weight and energy metabolism, including carbohydrate and fat metabolism.

The identification of AGT-117, AGT-110, AGT-199, AGT-107, AGT-114, AGT-116, AGT-115 and AGT-108 permits the generation of a range of therapeutic molecules capable of modulating expression of AGT-117, AGT-110, AGT-199, AGT-107, AGT-114, AGT-116, AGT-115 and AGT-108 or modulating the activity of AGT-117, AGT-110, AGT-199, AGT-107, AGT-114, AGT-116, AGT-115 and AGT-108. Modulators contemplated by the present invention includes agonists and antagonists of AGT-117, AGT-110, AGT-199, AGT-107, AGT-114, AGT-116, AGT-115 and AGT-108 expression. Antagonists of AGT-117, AGT-110, AGT-199, AGT-107, AGT-114, AGT-116, AGT-115 and AGT-108 expression include antisense molecules, ribozymes and co-suppression molecules.

Agonists include molecules which increase promoter activity or which interfere with negative regulatory mechanisms. Antagonists of AGT-117, AGT-110, AGT-199, AGT-107, AGT-114, AGT-116, AGT-115 and AGT-108 include antibodies and inhibitor peptide fragments. All such molecules may first need to be modified to enable such molecules to penetrate cell membranes. Alternatively, viral agents may be employed to introduce genetic elements to modulate expression of AGT-117, AGT-110, AGT-199, AGT-107, AGT-114, AGT-116, AGT-115 and AGT-108. In so far as AGT-117, AGT-110, AGT-199, AGT-107, AGT-114, AGT-116, AGT-115 and AGT-108 acts in association with other genes such as the ob gene which encodes leptin, the therapeutic molecules may target the AGT-117, AGT-110, AGT-199, AGT-107, AGT-114, AGT-116, AGT-115 and AGT-108 and ob genes or their translation products.

The present invention contemplates, therefore, a method for modulating expression of one or more of AGT-117, AGT-110, AGT-199, AGT-107, AGT-114, AGT-116, AGT-115 and AGT-108 in a mammal, said method comprising contacting the AGT-117, AGT-110, AGT-199, AGT-107, AGT-114, AGT-116, AGT-115 and AGT-108 gene with an effective amount of a modulator of AGT-117, AGT-110, AGT-199, AGT-107, AGT-114, AGT-116, AGT-115 and/or AGT-108 expression for a time and under conditions sufficient to up-regulate or down-regulate or otherwise modulate expression of AGT-117, AGT-110, AGT-199, AGT-107, AGT-114, AGT-116, AGT-115 and AGT-108. For example, a nucleic acid molecule encoding AGT-117, AGT-110, AGT-199, AGT-107, AGT-114, AGT-116, AGT-115 and AGT-108 or a derivative or homolog thereof may be introduced into a cell to enhance the ability of that cell to produce AGT-117, AGT-110, AGT-199, AGT-107, AGT-114, AGT-116, AGT-115 and AGT-108, conversely, AGT-117, AGT-110, AGT-199, AGT-107, AGT-114, AGT-116, AGT-115 and AGT-108 antisense sequences such as oligonucleotides may be introduced to decrease the availability of AGT-117, AGT-110, AGT-199, AGT-107, AGT-114, AGT-116, AGT-115 and AGT-108 molecules.

Another aspect of the present invention contemplates a method of modulating activity of AGT-117, AGT-110, AGT-199, AGT-107, AGT-114, AGT-116, AGT-115 and AGT-108 in a mammal, said method comprising administering to said mammal a modulating effective amount of a molecule for a time and under conditions sufficient to increase or decrease AGT-117, AGT-110, AGT-199, AGT-107, AGT-114, AGT-116, AGT-115 and AGT-108 activity. The molecule may be a proteinaceous molecule or a chemical entity and may also be a derivative of AGT-117, AGT-110, AGT-199, AGT-107, AGT-114, AGT-116, AGT-115 and AGT-108 or its ligand.

Modulating levels of AGT-117, AGT-110, AGT-199, AGT-107, AGT-114, AGT-116, AGT-115 and AGT-108 expression is important in the treatment of a range of conditions such as obesity, anorexia, energy imbalance, diabetes, metabolic syndrome, dyslipidemia, hypertension, insulin resistance and muscle development conditions. It may also be useful in the agricultural industry to assist in the generation of leaner animals, or where required, more obese animals. Accordingly, the mammal contemplated by the present invention includes but is not limited to humans, primates, livestock animals (e.g. pigs, sheep, cows, horses, donkeys), laboratory test animals (e.g. mice, rats, guinea pigs, hamsters, rabbits), companion animals (e.g. dogs, cats) and captured wild animals (e.g. foxes, kangaroos, deer). A particularly preferred host is a human, primate or livestock animal.

Accordingly, the present invention contemplates therapeutic and prophylactic uses of AGT-117, AGT-110, AGT-199, AGT-107, AGT-114, AGT-116, AGT-115 and/or AGT-108 amino acid and nucleic acid molecules in addition to AGT-117, AGT-110, AGT-199, AGT-107, AGT-114, AGT-116, AGT-115 and/or AGT-108 agonistic and antagonistic agents.

The present invention contemplates, therefore, a method of modulating expression of AGT-117, AGT-110, AGT-199, AGT-107, AGT-114, AGT-116, AGT-115 and/or AGT-108 in a mammal, said method comprising contacting the AGT-117, AGT-110, AGT-199, AGT-107, AGT-114, AGT-116, AGT-115 and/or AGT-108 genes with an effective amount of an agent for a time and under conditions sufficient to up-regulate, down-regulate or otherwise module expression of AGT-117, AGT-110, AGT-199, AGT-107, AGT-114, AGT-116, AGT-115 and/or AGT-108. For example, antisense sequences such as oligonucleotides may be utilized. Alternatively, sense molecules may be employed to induce co-suppression and/or RNAi.

Conversely, nucleic acid molecules encoding AGT-117, AGT-110, AGT-199, AGT-107, AGT-114, AGT-116, AGT-115 and/or AGT-108 or derivatives thereof may be introduced to up-regulate one or more specific functional activities.

Another aspect of the present invention contemplates a method of modulating activity of AGT-117, AGT-110, AGT-199, AGT-107, AGT-114, AGT-116, AGT-115 and/or AGT-108 in a subject, said method comprising administering to said subject a modulating effective amount of an agent for a time and under conditions sufficient to increase or decrease AGT-117, AGT-110, AGT-199, AGT-107, AGT-114, AGT-116, AGT-115 and/or AGT-108 activity.

Modulation of said activity by the administration of an agent to a mammal can be achieved by one of several techniques, including but in no way limited to introducing into said mammal a proteinaceous or non-proteinaceous molecule which:

(i) modulates expression of AGT-117, AGT-110, AGT-199, AGT-107, AGT-114, AGT-116, AGT-115 and/or AGT-108;

(ii) functions as an antagonist of AGT-117, AGT-110, AGT-199, AGT-107, AGT-114, AGT-116, AGT-115 and/or AGT-108;

(iii) functions as an agonist of AGT-117, AGT-110, AGT-199, AGT-107, AGT-114, AGT-116, AGT-115 and/or AGT-108.

The proteinaceous molecule may be derived from natural or recombinant sources including fusion proteins or following, for example, natural product screening. The non-proteinaceous molecule may be, for example, a nucleic acid molecule or may be derived from natural sources, such as for example natural product screening or may be chemically synthesized. The present invention contemplates chemical analogs of AGT-117, AGT-110, AGT-199, AGT-107, AGT-114, AGT-116, AGT-115 and/or AGT-108 or small molecules capable of acting as agonists or antagonists. Chemical agonists may not necessarily be derived from AGT-117, AGT-110, AGT-199, AGT-107, AGT-114, AGT-116, AGT-115 and/or AGT-108 but may share certain conformational similarities. Alternatively, chemical agonists may be specifically designed to mimic certain physiochemical properties. Antagonists may be any compound capable of blocking, inhibiting or otherwise preventing AGT-117, AGT-110, AGT-199, AGT-107, AGT-114, AGT-116, AGT-115 and/or AGT-108 from carrying out their normal biological functions. Antagonists include monoclonal antibodies and antisense nucleic acids which prevent transcription or translation of AGT-117, AGT-110, AGT-199, AGT-107, AGT-114, AGT-116, AGT-115 and/or AGT-108 genes or mRNA in mammalian cells. Modulation of expression may also be achieved utilizing antigens, RNA, ribosomes, DNAzymes, RNA aptamers or antibodies.

The proteinaceous or non-proteinaceous molecule may act either directly or indirectly to modulate the expression of AGT-117, AGT-110, AGT-199, AGT-107, AGT-114, AGT-116, AGT-115 and/or AGT-108 or the activity of AGT-117, AGT-110, AGT-199, AGT-107, AGT-114, AGT-116, AGT-115 and/or AGT-108. The molecule acts directly if it associates with AGT-117, AGT-110, AGT-199, AGT-107, AGT-114, AGT-116, AGT-115 and/or AGT-108 or AGT-117, AGT-110, AGT-199, AGT-107, AGT-114, AGT-116, AGT-115 and/or AGT-108 to modulate expression or activity. The molecule acts indirectly if it associates with a molecule other than AGT-117, AGT-110, AGT-199, AGT-107, AGT-114, AGT-116, AGT-115 and/or AGT-108 or AGT-117, AGT-110, AGT-199, AGT-107, AGT-114, AGT-116, AGT-115 and/or AGT-108 which other molecule either directly or indirectly modulates the expression or activity of AGT-117, AGT-110, AGT-199, AGT-107, AGT-114, AGT-116, AGT-115 and/or AGT-108 or AGT-117, AGT-110, AGT-199, AGT-107, AGT-114, AGT-116, AGT-115 and/or AGT-108. Accordingly, the method of the present invention encompasses the regulation of AGT-117, AGT-110, AGT-199, AGT-107, AGT-114, AGT-116, AGT-115 and/or AGT-108 or AGT-117, AGT-10, AGT-199, AGT-107, AGT-114, AGT-116, AGT-115 and/or AGT-108 expression or activity via the induction of a cascade of regulatory steps.

The molecules which may be administered to a mammal in accordance with the present invention may also be linked to a targeting means such as a monoclonal antibody, which provides specific delivery of these molecules to the target cells.

A further aspect of the present invention relates to the use of the invention in relation to mammalian disease conditions. For example, the present invention is particularly useful but in no way limited to use in a therapeutic or prophylactic treatment of obesity, anorexia, diabetes or energy imbalance.

Accordingly, another aspect of the present invention relates to a method of treating a mammal suffering from a condition characterized by one or more symptoms of obesity, anorexia, diabetes and/or energy imbalance, said method comprising administering to said mammal an effective amount of an agent for a time and under conditions sufficient to modulate the expression of AGT-117, AGT-110, AGT-199, AGT-107, AGT-114, AGT-116, AGT-115 and/or AGT-108 or sufficient to modulate the activity of AGT-117, AGT-110, AGT-199, AGT-107, AGT-114, AGT-116, AGT-115 and/or AGT-108.

In another aspect, the present invention relates to a method of treating a mammal suffering from a disease condition characterized by one or more symptoms of obesity, anorexia, diabetes or energy imbalance, said method comprising administering to said mammal an effective amount of AGT-117, AGT-110, AGT-199, AGT-107, AGT-114, AGT-116, AGT-115 and/or AGT-108 or AGT-117, AGT-110, AGT-199, AGT-107, AGT-114, AGT-116, AGT-115 and/or AGT-108.

An “effective amount” means an amount necessary at least partly to attain the desired immune response, or to delay the onset or inhibit progression or halt altogether, the onset or progression of a particular condition of the individual to be treated, the taxonomic group of the individual to be treated, the degree of protection desired, the formulation of the vaccine, the assessment of the medical situation, and other relevant factors. It is expected that the amount will fall in a relatively broad range that can be determined through routine trials.

In accordance with these methods, AGT-117, AGT-110, AGT-199, AGT-107, AGT-114, AGT-116, AGT-115 and/or AGT-108 or AGT-117, AGT-110, AGT-199, AGT-107, AGT-114, AGT-116, AGT-115 and/or AGT-108 or agents capable of modulating the expression or activity of said molecules may be co-administered with one or more other compounds or other molecules. By “co-administered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

In yet another aspect, the present invention relates to the use of an agent capable of modulating the expression of AGT-117, AGT-110, AGT-199, AGT-107, AGT-114, AGT-116, AGT-115 and/or AGT-108 or a derivative, homolog or analog thereof in the manufacture of a medicament for the treatment of a condition characterized by obesity, anorexia, diabetes and/or energy imbalance.

In still yet another aspect, the present invention relates to the use of an agent capable of modulating the activity of AGT-117, AGT-110, AGT-199, AGT-107, AGT-114, AGT-116, AGT-115 and/or AGT-108 or a derivative, homolog, analog, chemical equivalent or mimetic thereof in the manufacture of a medicament for the treatment of a condition characterized by obesity, anorexia, diabetes and/or energy imbalance.

A further aspect of the present invention relates to the use of AGT-117, AGT-110, AGT-199, AGT-107, AGT-114, AGT-116, AGT-115 and/or AGT-108 or derivative, homolog or analog thereof or AGT-117, AGT-110, AGT-199, AGT-107, AGT-114, AGT-116, AGT-115 and/or AGT-108 or derivative, homolog, analog, chemical equivalent or mimetic thereof in the manufacture of a medicament for the treatment of a condition characterized by obesity, anorexia, diabetes, impaired muscle development and/or energy imbalance.

Still yet another aspect of the present invention relates to agents for use in modulating the expression of AGT-117, AGT-110, AGT-199, AGT-107, AGT-114, AGT-116, AGT-115 and/or AGT-108 or a derivative, homolog or analog thereof.

A further aspect relates to agents for use in modulating AGT-117, AGT-110, AGT-199, AGT-107, AGT-114, AGT-116, AGT-115 and/or AGT-108 activity or a derivative, homolog, analog, chemical equivalent or mimetic thereof.

Still another aspect of the present invention relates to AGT-117, AGT-110, AGT-199, AGT-107, AGT-114, AGT-116, AGT-115 and/or AGT-108 or derivative, homolog or analog thereof or AGT-117, AGT-110, AGT-199, AGT-107, AGT-114, AGT-116, AGT-115 and/or AGT-108 or derivative, homolog, analog, chemical equivalent or mimetic thereof for use in treating a condition characterized by one or more symptoms of obesity, anorexia, diabetes, impaired muscle development and/or energy imbalance.

In a related aspect of the present invention, the mammal undergoing treatment may be a human or an animal in need of therapeutic or prophylactic treatment.

Accordingly, the present invention contemplates in one embodiment a composition comprising a modulator of AGT-117, AGT-110, AGT-199, AGT-107, AGT-114, AGT-116, AGT-115 and AGT-108 expression or AGT-117, AGT-110, AGT-199, AGT-107, AGT-114, AGT-116, AGT-115 and AGT-108 activity and one or more pharmaceutically acceptable carriers and/or diluents. In another embodiment, the composition comprises AGT-117, AGT-110, AGT-199, AGT-107, AGT-114, AGT-116, AGT-115 and AGT-108 or a derivative, homolog, analog or mimetic thereof and one or more pharmaceutically acceptable carriers and/or diluents. The compositions may also comprise leptin or modulations of leptin activity or ob expression.

For brevity, all such components of such a composition are referred to as “active components”.

The compositions of active components in a form suitable for injectable use include sterile aqueous solutions (where water soluble) and sterile powders for the extemporaneous preparation of sterile injectable solutions. In all cases, the form must be sterile and must be fluid to the extent that easy syringability exists. It must be stable under the conditions of manufacture and storage and must be preserved against the contaminating action of microorganisms such as bacteria and fungi.

The carrier can be a solvent or other medium containing, for example, water, ethanol, polyol (for example, glycerol, propylene glycol and liquid polyethylene glycol, and the like), suitable mixtures thereof, and vegetable oils.

The preventions of the action of microorganisms can be brought about by various antibacterial and antifungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active components in the required amount in the appropriate solvent with optionally other ingredients, as required, followed by sterilization by, for example, filter sterilization, irradiation or other convenient means. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When AGT-117, AGT-110, AGT-199, AGT-107, AGT-114, AGT-116, AGT-115 and AGT-108 and AGT-117, AGT-110, AGT-199, AGT-107, AGT-114, AGT-116, AGT-115 and AGT-108 including AGT-117, AGT-110, AGT-199, AGT-107, AGT-114, AGT-116, AGT-115 and AGT-108 itself are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active component may be compounded for convenient and effective administration in sufficient amounts with a suitable pharmaceutically acceptable carrier in dosage unit form. A unit dosage form can, for example, contain the principal active component in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients.

In general terms, effective amounts of AGT-117, AGT-110, AGT-199, AGT-107, AGT-114, AGT-116, AGT-115 and AGT-108 will range from 0.01 ng/kg/body weight to above 10,000 mg/kg/body weight. Alternative amounts range from 0.1 ng/kg/body weight is above 1000 mg/kg/body weight. AGT-117, AGT-110, AGT-199, AGT-107, AGT-114, AGT-116, AGT-115 and AGT-108 may be administered per minute, hour, day, week, month or year depending on the condition being treated. The route of administration may vary and includes intravenous, intraperitoneal, sub-cutaneous, intramuscular, intranasal, via suppository, via infusion, via drip, orally or via other convenient means.

The pharmaceutical composition may also comprise genetic molecules such as a vector capable of transfecting target cells where the vector carries a nucleic acid molecule capable of modulating AGT-117, AGT-110, AGT-199, AGT-107, AGT-114, AGT-116, AGT-115 and AGT-108 expression or AGT-117, AGT-110, AGT-199, AGT-107, AGT-114, AGT-116, AGT-115 and AGT-108 activity. The vector may, for example, be a viral vector.

Still another aspect of the present invention is directed to antibodies to AGT-117, AGT-110, AGT-199, AGT-107, AGT-114, AGT-116, AGT-115 and AGT-108 and their derivatives and homologs. Such antibodies may be monoclonal or polyclonal and may be selected from naturally occurring antibodies to AGT-117, AGT-110, AGT-199, AGT-107, AGT-114, AGT-116, AGT-115 and AGT-108 or may be specifically raised to AGT-117, AGT-110, AGT-199, AGT-107, AGT-114, AGT-116, AGT-115 and AGT-108 or derivatives or homologs thereof. In the case of the latter, AGT-117, AGT-110, AGT-199, AGT-107, AGT-114, AGT-116, AGT-115 and AGT-108 or their derivatives or homologs may first need to be associated with a carrier molecule. The antibodies and/or recombinant AGT-117, AGT-110, AGT-199, AGT-107, AGT-114, AGT-116, AGT-115 and AGT-108 or their derivatives of the present invention are particularly useful as therapeutic or diagnostic agents.

For example, AGT-117, AGT-110, AGT-199, AGT-107, AGT-114, AGT-116, AGT-115 and AGT-108 and their derivatives can be used to screen for naturally occurring antibodies to AGT-117, AGT-110, AGT-199, AGT-107, AGT-114, AGT-116, AGT-115 and AGT-108 which may occur in certain autoimmune diseases or where cell death is occurring. These may occur, for example in some autoimmune diseases. Alternatively, specific antibodies can be used to screen for AGT-117, AGT-110, AGT-199, AGT-107, AGT-114, AGT-116, AGT-115 and AGT-108. Techniques for such assays are well known in the art and include, for example, sandwich assays and ELISA.

Antibodies to AGT-117, AGT-110, AGT-199, AGT-107, AGT-114, AGT-116, AGT-115 and AGT-108 of the present invention may be monoclonal or polyclonal and may be selected from naturally occurring antibodies to the AGT-117, AGT-110, AGT-199, AGT-107, AGT-114, AGT-116, AGT-115 and AGT-108 or may be specifically raised to the AGT-117, AGT-110, AGT-199, AGT-107, AGT-114, AGT-116, AGT-115 and AGT-108 or their derivatives. In the case of the latter, the AGT-117, AGT-110, AGT-199, AGT-107, AGT-114, AGT-116, AGT-115 and AGT-108 protein may need first to be associated with a carrier molecule. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies. The antibodies of this aspect of the present invention are particularly useful for immunotherapy and may also be used as a diagnostic tool or as a means for purifying AGT-117, AGT-110, AGT-199, AGT-107, AGT-114, AGT-116, AGT-115 and AGT-108.

For example, specific antibodies can be used to screen for AGT-117, AGT-110, AGT-199, AGT-107, AGT-114, AGT-116, AGT-115 and AGT-108 proteins. The latter would be important, for example, as a means for screening for levels of AGT-117, AGT-110, AGT-199, AGT-107, AGT-114, AGT-116, AGT-115 and AGT-108 in a cell extract or other biological fluid or purifying AGT-117, AGT-110, AGT-199, AGT-107, AGT-114, AGT-116, AGT-115 and AGT-108 made by recombinant means from culture supernatant fluid. Techniques for the assays contemplated herein are known in the art and include, for example, sandwich assays and ELISA.

It is within the scope of this invention to include any second antibodies (monoclonal, polyclonal or fragments of antibodies) directed to the first mentioned antibodies discussed above. Both the first and second antibodies may be used in detection assays or a first antibody may be used with a commercially available anti-immunoglobulin antibody. An antibody as contemplated herein includes any antibody specific to any region of AGT-117, AGT-110, AGT-199, AGT-107, AGT-114, AGT-116, AGT-115 and AGT-108.

Both polyclonal and monoclonal antibodies are obtainable by immunization with the enzyme or protein and either type is utilizable for immunoassays. The methods of obtaining both types of sera are well known in the art. Polyclonal sera are less preferred but are relatively easily prepared by injection of a suitable laboratory animal with an effective amount of AGT-117, AGT-110, AGT-199, AGT-107, AGT-114, AGT-116, AGT-115 and AGT-108, or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example, Douillard and Hoffman, Basic Facts about Hybridomas, in Compendium of Immunology Vol. 11, ed. by Schwartz, 1981; Kohler and Milstein, Nature 256: 495-499, 1975; Kohler and Milstein, European Journal of immunology 6: 511-519, 1976).

Another aspect of the present invention contemplates a method for detecting AGT-117, AGT-110, AGT-199, AGT-107, AGT-114, AGT-116, AGT-115 and AGT-108 or a derivative or homolog thereof in a biological sample from a subject, said method comprising contacting said biological sample with an antibody specific for AGT-117, AGT-110, AGT-199, AGT-107, AGT-114, AGT-116, AGT-115 and AGT-108 or their antigenic derivatives or homologs for a time and under conditions sufficient for a complex to form, and then detecting said complex.

The presence of the complex is indicative of the presence of AGT-117, AGT-110, AGT-199, AGT-107, AGT-114, AGT-116, AGT-115 and AGT-108. This assay may be quantitated or semi-quantitated to determine a propensity to develop obesity or other conditions or to monitor a therapeutic regimum.

The presence of AGT-117, AGT-110, AGT-199, AGT-107, AGT-114, AGT-116, AGT-115 and AGT-108 may be accomplished in a number of ways such as by Western blotting and ELISA procedures. A wide range of immunoassay techniques are available as can be seen by reference to U.S. Pat. Nos. 4,016,043, 4,424,279 and 4,018,653. These, of course, includes both single-site and two-site or “sandwich” assays of the non-competitive types, as well as in the traditional competitive binding assays. These assays also include direct binding of a labelled antibody to a target.

Sandwich assays are among the most useful and commonly used assays. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-AGT-117, AGT-110, AGT-199, AGT-107, AGT-114, AGT-116, AGT-115 and AGT-108 complex, a second antibody specific to the AGT-117, AGT-110, AGT-199, AGT-107, AGT-114, AGT-116, AGT-115 and AGT-108, labelled with a reporter molecule capable of producing a detectable signal, is then added and incubated, allowing time sufficient for the formation of another complex of antibody-AGT-117, AGT-110, AGT-199, AGT-107, AGT-114, AGT-116, AGT-115 and AGT-108-labelled antibody. Any unreacted material is washed away, and the presence of AGT-117, AGT-110, AGT-199, AGT-107, AGT-114, AGT-116, AGT-115 and AGT-108 is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent. In accordance with the present invention, the sample is one which might contain AGT-117, AGT-110, AGT-199, AGT-107, AGT-114, AGT-116, AGT-115 and AGT-108 including cell extract, tissue biopsy or possibly serum, saliva, mucosal secretions, lymph, tissue fluid and respiratory fluid. The sample is, therefore, generally a biological sample comprising biological fluid but also extends to fermentation fluid and supernatant fluid such as from a cell culture.

The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to about 37° C.) to allow binding of any subunit present in the antibody. Following the incubation period, the antibody subunit solid phase is washed and dried and incubated with a second antibody specific for a portion of AGT-117, AGT-110, AGT-199, AGT-107, AGT-114, AGT-116, AGT-115 and AGT-108. The second antibody is linked to a reporter molecule which is used to indicate the binding of the second antibody to AGT-117, AGT-110, AGT-199, AGT-107, AGT-114, AGT-116, AGT-115 and AGT-108.

An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. A “reporter molecule” also extends to use of cell agglutination or inhibition of agglutination such as red blood cells on latex beads, and the like.

Alternately, fluorescent compounds, such as fluorecein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength the fluorescence observed indicates the presence of the hapten of interest. Immunofluorescene and EIA techniques are both very well established in the art and are particularly preferred for the present method.

However, other reporter molecules, such as radioisotope, chemiluminescent or bioluminescent molecules, may also be employed.

The present invention also contemplates genetic assays such as involving PCR analysis to detect AGT-117, AGT-110, AGT-199, AGT-107, AGT-114, AGT-116, AGT-115 and AGT-108 or their derivatives.

The assays of the present invention may also extend to measuring AGT-117, AGT-110, AGT-199, AGT-107, AGT-114, AGT-116, AGT-115 and AGT-108 or AGT-117, AGT-110, AGT-199, AGT-107, AGT-114, AGT-116, AGT-115 and AGT-108 in association with ob or leptin.

The present invention is further described by the following non-limiting Examples.

### EXAMPLE 1

### Partial sequence of Psammomys Obesus AGT-117

AGT-117 was identified by different differential display of fed and fasted diabetic and non-diabetic Psammomys obesus liver cDNA.

The partial nucleotide sequence is as follows:—

| | |
|---|---|
| ATGGATGTAGACTTNGGTAATTTGGATTATACAAACAACTAAACGTTTTAAGCAGAATGAG | [SEQ ID NO: 1] |
| TAATGGATCATAATAATAGAATCATGGTGCTGAGGGTGATTTGAACTGTGGGACCCTGTCT | |
| CAAGAGGTTTCAGGGAGAAGAATATTAGTATGAGACCTAGGGACTGTTGTGATAGTTTGGT | |
| GAAGAATGTGACTGTTTTCTGCCCTTGTCCAAAAAAAAAAA. | |

### EXAMPLE 2

### AGT-117 Gene Expression

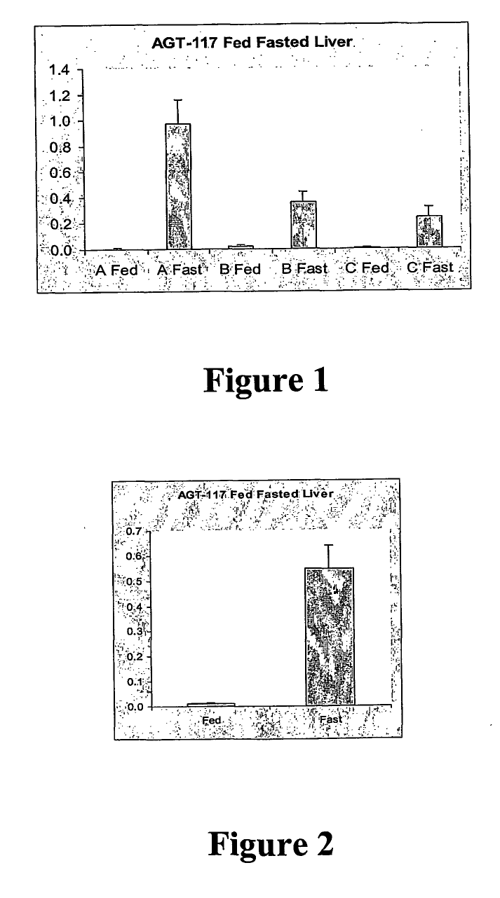

SYBR green real-time (RT)-PCR on liver cDNA from A, 13, and C fed and fasted animals showed that AGT-117 expression was significantly increased with fasting in A (p<0.001), B (p<0.001) and C animals (p<0.001), compared to fed control animals (n=≧5 animals in each group, FIG. 1).

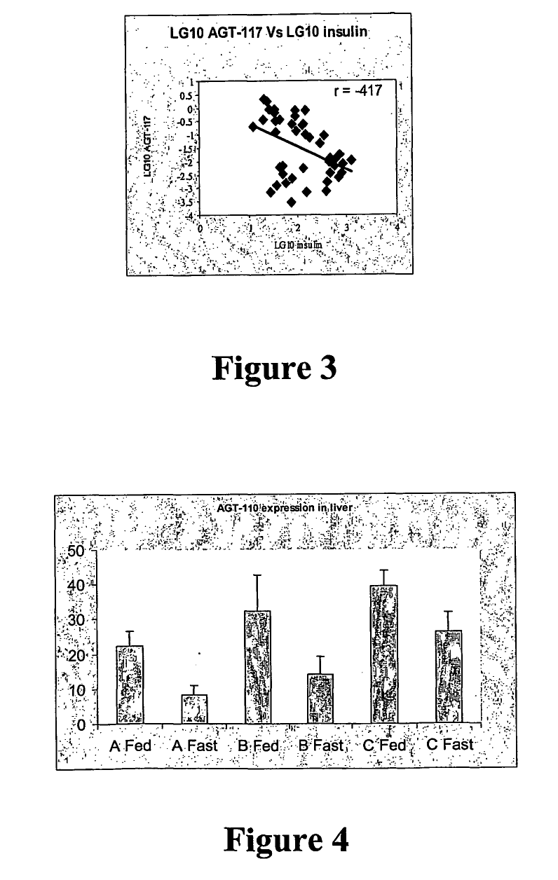

In the fed state, there was a significant decrease in expression between A and B (p=0.013) and A and C (p=0.037) but not between B and C fed animals (FIG. 1). In the fasted state the only significant difference was between A and C (p=0.047) animals. When animal groups were pooled there was a significant decrease in AGT-117 expression with fasting (p<0.001, FIG. 2). AGT-117 expression negatively correlated with log plasma isulin (all animals, p=0.005, FIG. 3) but did not correlate with bodyweight or plasma glucose.

### EXAMPLE 3

### AGT-117 Gene Homology

Blast searches with the partial sequence for AGT-117 revealed 93% homology with a mouse cDNA sequence from clone 601668821F1 NCI_CGAP_IMAGE:3968741 5′ mRNA sequence and 90% homology with a BAC cDNA clone, mouse BAC-146N21 of chromosome X that contains iduronate-2-sulfatase gene; complete sequence. AGT-117 is currently undergoing RACE PCR to obtain more sequence.

### EXAMPLE 4

### Partial Sequence of Psammomys Obesus AGT-110

AGT-110 was identified by differential display of fed and fasted diabetic and non-diabetic Psammomys obesus liver cDNA.

The partial nucleotide sequence is as follows:—

| | |
|---|---|
| GACGTAGAGCCGAGCGCCGAGCTCTCAACACCCCAGCCTCCCTCAGCCATTTATTTATTC | [SEQ ID NO: 2] |
| CTGTCCCGCCTCAGCACTCAGCAGTGAGCTTGAAATAAAGGCAACTTTCTTGTTTTCAAA | |
| AAAAAAAA. | |
| CACAGGACGAAAGGCACCATGGCACTGAGCACTCAGACCCAGGCTGCCTGTCTCCTGCTG | [SEQ ID NO: 28] |
| CTTCTCATTGCCAGCCTGAGCAGTGGTGCCATTCTCCAGCAACAGCTCGGACAGCCCGCA | |
| GCGCTCCAGCCGTGGCACAGGGCAGAATCCAGTGCCGACAGGATGCTGATCCAGACACGA | |
| AAGAAGCGTGACACACACTTCCCCACCTGCATATTCTGCTGTCATTGCTGTAAGAATCCT | |
| GGCTGCGGACTGTGCTGCAAGACGTAGAGCCGAGCGCCGACCTCTCAACACCCCAGCCTC | |
| CCTCAGCCATTTATTTATTCCTGTCCCGCCTCAGCCT. | |

### EXAMPLE 5

### AGT-110 Gene Expression

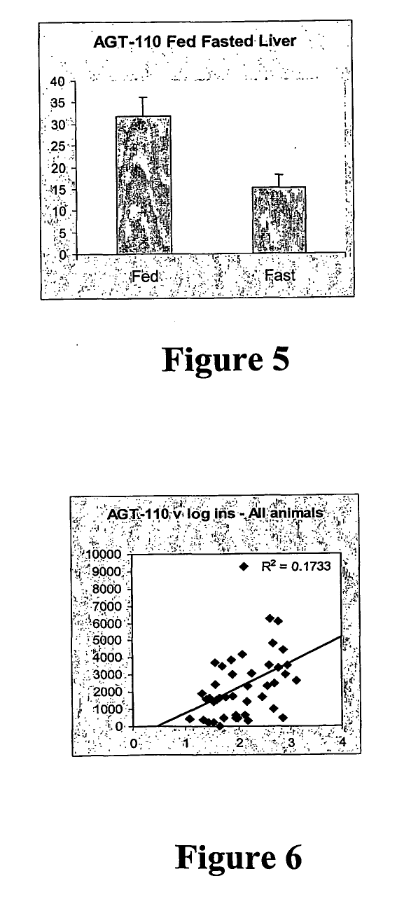

SYBR green RT-PCR on liver cDNA from A, B, and C fed and fasted animals showed that there were no significant differences between A, B, or C groups in either fed or fasted states (n≧5 animals in each group, FIG. 4). When animal groups were pooled there was a significant decrease in AGT-110 expression with fasting (p=0.003, FIG. 5). AGT-110 expression positively correlated with log plasma insulin (all animals, p=0.008, FIG. 6) but did not correlate with bodyweight or plasma glucose. AGT-110 was expressed predominantly in the liver with lower amounts detected in the cerebellum, subscapular fat and adrenal gland.

Real time PCR of a variety of tissues from Psammomys obesus showed that AGT-110 was found to be expressed predominately in liver, with small amounts detected in cerebellum, subscapular fat and adrenal gland.

### EXAMPLE 6

### AGT-110 Gene Homology

AGT-110 nucleotide sequence has strong homology to mouse, rat and human hepcidin antimicrobial peptide (HAMP). Human hepcidin (AGT-110) is 391 base pairs in length and encodes a protein amino acids 84 long. Hepcidin antimicrobial peptide is a disulfide-bonded peptide exhibiting antimicrobial activity. Hepcidin may act as a signaling molecule involved in the maintenance of iron homeostasis and have additional functions distinct from its antimicrobial activity (pigeon et al., J. Biol. Chem. 276(11): 7811-7819, 2001). Any alterations in hepcidin level or activity may affect the liver's role in glucose, fat and amino acid metabolism and may contribute to the development of obesity and type 2 diabetes.

Hepcidin is synthesized in the liver in the form of a propeptide that contains 83 amino acids and is converted into mature peptides of 20, 22 and 25 amino acids (Park et al., J.

Biol. Chem. 276: 7806-7610, 2001). The murine precursor protein prohepcidin is exclusively localized in the nucleus, the resulting protein is found in the cytoplasm (Pigeon et al., 2001, supra).

Hepcidin may play a specific role in iron overload. It is overexpressed in livers of experimentally (carbonyl iron and iron-dextran-treated mice) and spontaneously (β₂-microglobulin knockout mice) iron overloaded mice (Pigeon et al., 2001, supra). The murine hepcidin has strong homology in its C-terminal region to human hepcidin. Both mouse and human genes have 3 exons and 2 introns and are located on chromosome 7 and 19, respectively (Pigeon et al., 2001, supra). Hepcidin mRNA is predominately expressed in mouse and human liver. Hepcidin has been purified from human blood ultrafiltrate and from urine (Park et al., 2001, supra; Krause et al., FEBS Lett. 480: 147-150, 2000).

Bioinformatics analysis predicted a signal peptide in the sequence with the most likely cleavage site between amino acid 24 and 25 (TSG-SV). One ER membrane retention signal (XXRR-like motif in the C-terminus: MCCK) and one prenylation motif (CC motif near the C-terminus: CCKT) was also predicted. One possible serine phosphorylation site (48) and one threonine site (61) was found. A cysteine-rich domain from amino acids 66-82 was found.

Human hepcidin is located on chromosome 19 and has been mapped to the interval 19q13.1. There are several known human obesity QTLs in this region:

19q13.1-q13.2: LIPE lipase, hormone sensitive, phenotype: Obesity (Magré et al., Diabetes 47: 284-286, 1998)

19p13.3: INSR insulin receptor, phenotype: Obesity (BMI>26) in hypertensives (Zee et al., J. Hypertension 12: S13-S22, 1994)

19p13.2: LDLR low-density lipoprotein receptor, phenotype: BMI in hypertensives (Zee et al., Biochem Biophys Res Commun. 189: 965-971, 1992; Zee et al., Clin Genet. 47: 118-121, 1995; Griffiths et al., Clin Exp Pharmacol Physiol. 22: 496-498, 1995) and Obesity (BMI>26) (Rutherford et al., Int J Obes. 21: 1032-1037, 1997).

### EXAMPLE 7

### Partial Sequence of Psammomys Obesus AGT-199

AGT-199 was identified by differential display of fed and fasted diabetic and non-diabetic Psammomys obesus liver cDNA.

The partial nucleotide sequence is as follows:—

| | |
|---|---|
| TCATTTACTGGTCTACATGTCTGTTTTGGTGGCAATATTACATTGTTTTTGTAACAGTGG | [SEQ ID NO: 3] |
| TTCTGTAGTGTCCTTTGAAATCAAGTGTTCTTATAACTCCAAAAAAAAAAA. | |

### EXAMPLE 8

### AGT-199 Gene Expression

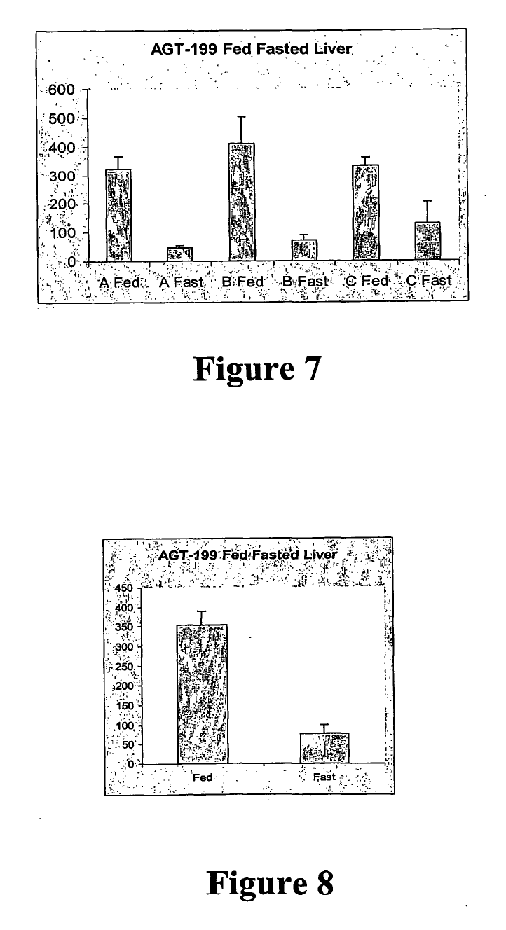

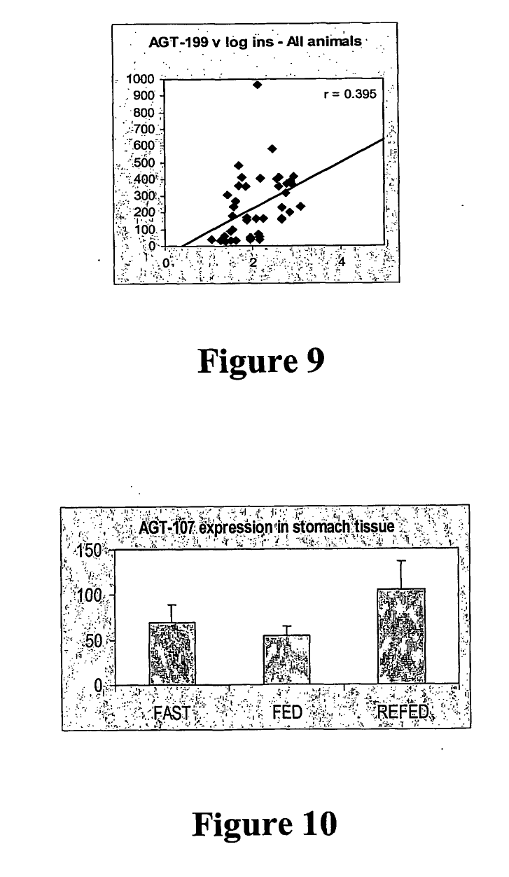

SYBR green RT-PCR on liver cDNA from A, B, and C fed and fasted animals showed that AGT-199 expression was significantly reduced with fasting in A (p=0.001) and C animals (p=0.001), while there was a strong trend for reduced expression with fasting in group B (p=0.065) (n≧5 animals in each group, FIG. 7). There were no significant differences between A, B, or C groups in either fed or fasted states. When animal groups were pooled there was a significant decrease in AGT-199 expression with fasting (p=0.001, FIG. 8). AGT-199 expression positively correlated with log plasma insulin (all animals, p=0.009, FIG. 9) but did not correlate with bodyweight or plasma glucose.

### EXAMPLE 9

### AGT-199 Gene Homology

Blast searches with the partial sequence of AGT-199 have not identified any EST's or known genes. AGT-199 is currently undergoing RACE PCR to obtain more sequence.

### EXAMPLE 10

### Partial Sequence of Psammomys Obesus AGT-107

AGT-107 was identified by differential display of fasted, fed and re-fed stomach cDNA from Psammomys obesus.

The partial nucleotide sequence is as follows:—

| | |
|---|---|
| CAGAAAAAAGTGAAAGAAAAGCTCCATGCAGTTAACGATGAAGAGTGCACTACCCTAAAA | [SEQ ID NO: 4] |
| GCAGGATGGCTGTCAGAAGAATGCATCAATGCAATCATGAGCTTCGTGTCCAGAAAAGCA | |
| AAGCTGTGAAGACCCACCACAGCAGCTAGACATCTCAGAGGAAGAATGTGCTGTGAGTTC | |
| CAGTTTGGGATACTTGAATGACACAAACTCCACTGTGCCTTTCCCTTGATTAACAGAGCA | |
| ATTTCGATGAGAATGCTTTACAGCACTGACAAATAAAAACTTTCATAAATCTAAAAAAAA | |
| AAA. | |

### EXAMPLE 11

### AGT-107 Gene Expression

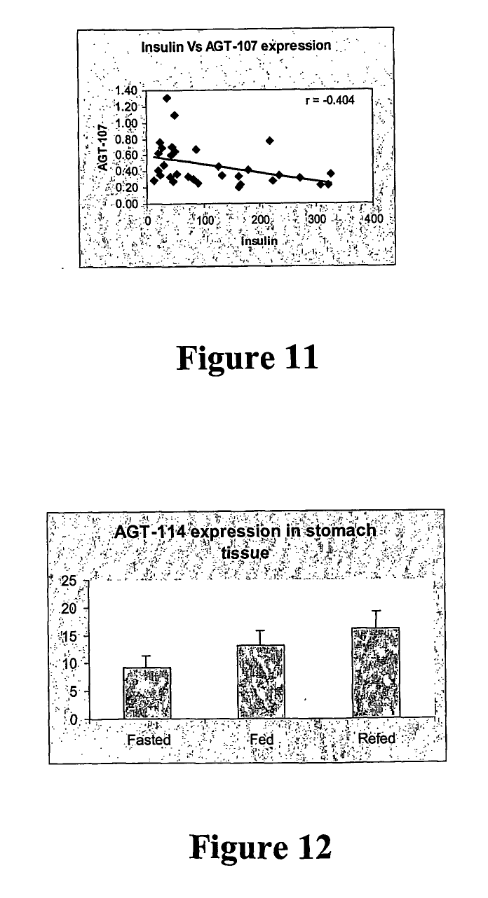

SYBR green RT-PCR on stomach cDNA from fasted, fed and re-fed animals showed that gene expression was only significantly higher in fasted compared to fed animals (p=0.003, FIG. 10, n≧13 in each group). AGT-107 was negatively correlated with plasma insulin (p=0.022, FIG. 11). No correlations were found between AGT-107 and body weight, plasma glucose, stomach content or stomach weight (content removed).

### EXAMPLE 12

### AGT-107 Gene Homology

Blast searches with the partial sequence of AGT-107 gave 83% nucleotide homology with mouse peroxisomal Δ³, Δ²-enoyl-Coenzyme A isomerase mRNA.

The pathway of β-oxidation requires additional auxiliary enzymes for the complete metabolism of fatty acids, including peroxisomal Δ³, Δ²-enoyl-Coenzyme A isomerase (PECI) which is essential for the β-oxidation of unsaturated fatty acids (Geisbrecht et al., J. Biol. Chem. 274: 21797-21803, 1999; Geisbrecht et al., J. Biol. Chem. 273: 33184-33191, 1998; Gurvitz et al., J. Biol. Chem. 273: 31366-31374, 1998). PECI is a ubiquitously expressed mammalian Δ³, Δ²-enoyl-Coenzyme A isomerase which is homologous to the Δ³, Δ²-enoyl-Coenzyme A isomerase of yeast (Eci1p)(1).

In human multi-tissue Northern blots (CLONTECH), PECI mRNA has been detected in 15 tissues (heart, brain, placenta, lung, liver, skeletal muscle, kidney, pancreas, spleen, thymus, prostrate, testis, ovary, small intestine, colon) but appeared most abundant in heart, skeletal muscle, kidney, pancreas and liver (Geisbrecht et al., 1999, supra).

Previously characterized Δ³, Δ²-enoyl-Coenzyme A isomerases have been grouped into the hydratase/isomerase superfamily of acyl-CoA binding proteins (Muller-Newen et al., Eur. J. Biochem. 228: 68-73, 1995) and contain the sequence fingerprint VSXINGX₃AGGXLX₄CDY. However, human PECI lacks this motif (Geisbrecht et al., 1998, supra), but contains a conserved (mouse PECI and yeast Eci1p) NGPA(V/I)G(I/L)S motif (Geisbrecht et al., 1999, supra) absent from the superfamily. The significance of these structural differences is yet to be determined.

Because PECI is involved in fat oxidation, any disturbances in PECI level or activity could potentially lead to obesity and diabetes.

### EXAMPLE 13

### Partial Sequence of Psammomys Obesus AGT-114

AGT-114 was identified by differential display of fasted, fed and re-fed stomach cDNA from Psammomys obesus.

The partial sequence for AGT-114 is as follows:—

| | |
|---|---|
| CTGTCCATGGCTGGGGAAGGACCTCACCAACTGCCTGCATCTGGTCAAGGAAGAGAGTGA | [SEQ ID NO: 5] |
| AAAGGGGGAGGGTAGGAGAAGGCACCAGTGGTGGCAGCAACTGCTTGTTGTGCATGAGTC | |
| TTTCCCAAGGGAGTCCTGAGGCCCGGTCCCTGTTAGAGGGTGGGAAATCGGAAGTGGCTG | |
| CTGTGGTTGAGGTGAGCCCTCANAAGAGCTGGAGCAACCCCTCCCAAGGTCCCAGCACTG | |
| CTTCCAAAGAGCCCAGCAAACCCTGCTTTCCTACACACTTGAATGGAAAAAAAAAAAA. | |

A more complete sequence is provided below:—

| | |
|---|---|
| TCAGGGCGGGGAAGAAGATGCTAAAAACTATAAGCAATCAGCCCAGTAATGGATTTCTGT | [SEQ ID NO: 35] |
| CAGGAGAGTGAAACTGTTTTAGAAAATAATGAAAATAAGAAAATTGAAGACACAGAAGAA | |
| ACTGTGCTGACTTTAAGTTGTCCAGATGAGAGAAGCGAAAGGAATCACGTTTGCTGTCTT | |
| CTCAGTATCAGTGATCTCACGCTGAACGAGGATGAGCGGGCCAGCGAGTTTGCCATCAAC | |
| ACTGGATGGGAGGGAGCTGTCCATGGCTGGGGAAGGACCTCACCAACTGCCTGCATCTGG | |
| TCAAGGAAGAGAGTGAAAAGGGGGAGGGTAGGAGAAGGCACCAGTGGTGGCAGCAACTGC | |
| TTGTTGTGCATGAGTCTTTCCCAAGGGAGTCCTGAGGCCCGGTCCCTGTTAGAGGGTGGG | |
| AAATCGGAAGTGGCTGCTGTGGTTGAGGTGAGCCCTCANAAGAGCTGGAGCAACCCCTCC | |
| CAAGGTCCCAGCACTGCTTCCAAAGAGCCCAGCAAACCCTGCTTTCCTACACACTTGAAT | |
| GGAAAAAAAAAAAA | |

A translation from nucleotide 34 to 551 of SEQ D NO:35 gives the following amino acid sequence:—

| | |
|---|---|
| AISPVMDFCQESETVLENNENKKIEDTEETVLTLSCPDERSERNHVCCLLSISDLTLNEDE | [SEQ ID NO: 29] |
| RASEFAINTGWEGAVHGWGRTSPTACIWSRKRVKRGRVGEGTSGGSNCLLCMSLSQGSP1E | |
| ARSLLEGGKSEVAAVVEVSPXKSWSNPSQGPSTASKEPSKPCFPTHLNGKKK. | |

Corresponding similar sequences were identified in human and murine and as follows:—

| | |
|---|---|
| MDLCQKNETDLENAENNEIQFTEETEPTYTCPDGKSEKNHVYCLLDVSDITLEQDEKAKE | [SEQ ID NO: 30] |
| FIIGTGWEEAVQGWGRTSPAACIWPRKIPKKARVGEGACSDCLVCVNLSHWSLQTKPPTE | |
| GGPEKDQSSPSQTQAAPQGPSTASRAISDICFPTYFRAEKKSLQIKEFIWCNKDWAIPGT | |
| NRGKASGNPSGGAHRGLSIPGPLTSRALLVLPPLKALLSNALDVLGKKSKNSFLQSEEKV | |
| LDVEKDGCVAYAYGLKTADGKGEKRASELAKHPMVNDTPSSPSPAAQISLLTDPEQRCLH | |
| WSLLSEKNLACPPDPSNRYLAALQLLQKRGVQSYKSKFKAKEPRSPVITRKHVLPKAKQ | |
| ENRPQMLETKVFPRPVLPSLTVSRVIIPVSTHRIL. | |
| MDVCEESETFLENTENQKIEATEETAPTLHCPDEKSERSHVCCLLGVSDLTLEEDGRASEC | [SEQ ID NO: 31] |
| AISTGWEEAVHGWGRTSPTACIWSKKKVKRGRAREGTNGGNDCLFCMSLSQGSLEPRSLLE | |
| VGKLEAGAEAEVSTQKSWSSEKNWSGLSQGPGTASREQSNKLCIPTDVHGEKKSLQLKEFI | |
| WCMEEWPMPETVSSKAGRNPSGSPEQGLSTPDSLAAKALVVLPPLKSAPHNLDVLSKKSRN | |
| IFWQPEEKVLRVEKDDCMACADGLKGVDGKGEKRHFELASPVKVTNVLPFPPTAAQTHLLS | |
| AESQRCCLHWSLLPQKSTVFPPNPSDIHYLATLQVLGQQGKQSCRTRLKTKDTKPPRTTAK | |
| HIITEAKQQNRPHVLESKVFPKPLLPSLTVSRVVIPVSTHRVL. | |

A corresponding human AGT-114 nucleotide sequence is shown in SEQ ID NO:32:

| | |
|---|---|
| TTAAACAGCAAGAAGATGTTAAAAACTTTAAGCAAGCATCACAGTAATGGATCTCTGTCAG | (SEQ ID NO: 32] |
| AAAAATGAGACTGACTTAGAAAATGCTGAAAATAATGAAATTCAGTTCACAGAAGAAACAG | |
| AACCAACCTATACTTGTCCAGATGGAAAAAGTGAAAAAAATCATGTTTATTGTCTTCTCGA | |
| TGTCAGTGACATTACGCTTGAACAAGATGAAAAAGCCAAAGAGTTTATTATTGGAACTGGA | |
| TGGG. | |

### EXAMPLE 14

### AGT-114 Gene Expression

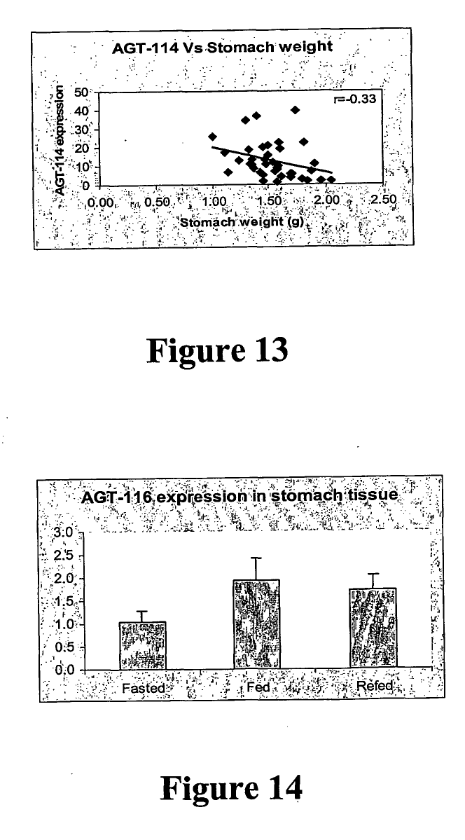

SYBR green RT-PCR on stomach cDNA from fasted, fed and re-fed animals showed that gene expression was significantly higher in re-fed when compared to fasted animals (p=0.037), but not compared to fed animals (n≧13 in each group, FIG. 12). AGT-114 expression was negatively correlated with stomach weight (content removed) (p=0.036, FIG. 13). No correlations were found between AGT-114 expression and stomach content, body weight, plasma glucose or insulin.

### EXAMPLE 15

### AGT-114 Gene Homology

Blast searches with the Psammomys obesus sequence found a match with a mouse testis cDNA, accession number AK006553, with a putative protein product of 409 amino acids.

### EXAMPLE 16

### Partial Sequence of Psammomys Obesus AGT-116

AGT-116 was identified by differential display of fasted, fed and re-fed stomach cDNA from Psammomys obesus.

The partial sequence for AGT-116 is as follows:—

| | |
|---|---|
| ACTGAGAGCTTCAGTGTTTATGTTATGAAAATAGAAAAAGCTGAATGCATTACACCCAGAG | [SEQ ID NO: 6] |
| CAAACTAGGAAGGAACTAATGAAGAATAAAAATTACTGAAATTATTAGAAAACAAAAACAA | |
| TAAAATTAACCAAAAGCTAATTCTTTGAAAATATATTAAATTGTCCCTTTGGCCCAATTGA | |
| TGAAAAAAAAAAA. | |

A more complete sequence for AGT-116 is shown in SEQ ID NO:33 below:

| | |
|---|---|
| AATTCGTTATATAAAAGTTAAAAAGAGAAGAGAAGAATCCAGGCACTGTAGCAGNNGGGNA | [SEQ ID NO: 33] |
| ATGTTTANTTNTAGGTGACTGCACACTTTGTGCCAGGGGNGCAAAACACAGAGCTTTGTTT | |
| TAATGCAAGGAGAAGGGGATGCTATCAGTACATTTATTTCCAGTTTGCTTTCTTGCCTTGT | |
| TTTCTTCTGNATTCCACTATACATCTACCAAGAATATAAAGGCACCAGGACTCCTGAACA | |
| CTCAGGCAATTTCCCCCAATTATCAGGCAGTATTAAAAACTAAAGCAGCCACAGTGAGATT | |
| CTACTTTACACTGGTGAGAATAGCTATCATAACAAATACATCAGTTTTTCTTTTTGTTCTG | |
| ATGNGTGATGAAAGAAGCCAAAGGAAAACNAGGCTTTGGCAAGAACATAAAAAAAGTTAGG | |
| AACGTTATANATTGCTAATGCAAATATGAATATTTGTCATTTCGTGAAGATTGGTGTTT | |
| TATCACAAAGGTTAAGTGTGGAATTGCATGTCNCNCATAGTATATATCAAAAAGAAATGAA | |
| AGCTGNTCCCAAACATTTTTCACAGATGTTTGTAGCAGGAGTAGTCATCAAAGCCAAAAGC | |
| TGGAAACCNCCTGAGTGTCTACCAGCAGATGATTGGAATAACCAATGGTAAATCAATATCT | |
| AAAACTTAACTATTCAGATAATAAGGTCTCATATAGTCCAGATTGGCCTAGAGCATCCCTC | |
| CTTTGATCTTCCCAAAGAGGGGATTCAGGAGGGAGAGTGTGACTGGGCANAGAGNAGGCNG | |
| GNCCTATGANCAAGATGTAAATTGTATTANTTAAAAAAAAAGATGACCTTGACTTCTGCTA | |
| CTCCTGCCCCTACCTACTGAGTGTTGGAATTACAGGCACACACCTCATCATGCGCATTCTT | |
| TAGTGCTGGTGATCAAACCCAGGGCTTCATGCATCCTAGCTAAGCACTCTACCAACTCAGC | |
| TATTTTTCAGCCCTAGCAATGTNCTTCTGAATGGCTCATGGGTCCAAGAGGAACTCAACAG | |
| AGAAATTATAGAATAGTTTACTTCCATGGAAATGAAACCTCAGCATCTNAGAATTGTGGGA | |
| TATTGCTGTATCAGAGGGAAGTTTCAAGCTTNGNGTGCTCCATTAGCAAACAGGAANGGAC | |
| CAGACTGAGAGCTTCAGTGTTTATGTTATGAAAATAGAAAAAGCTGAATGCATTACACCCA | |
| GAGCAAACTAGGAAGGAACTAATGAAGAATAAAAATTACTGAAATTATTAGAAAACAAAAA | |
| CAATAAAATTAACCAAAAGCTAATTCTTTGAAAATATATTAAATTGTCCCTTTGGCCCAAT | |
| TGATGAAAAAAAAAAA. | |

An even more complete nucleotide sequence is shown in SEQ ID NO:34:—

| | |
|---|---|
| CATAAAAAAAGTTAGGAACGTTATATATTGCTAATGCAAATATGAAATATTTGTCATTTCG | [SEQ ID NO: 34] |
| TGAAAGATTGGTGTTTTATCACAAAGGTTAAGTGTGGAATTGCATGTCGCACATAGTATAT | |
| ATCAAAAAGAAATGAAAGCTGNTCCCAAACATTTTTCACAGATGTTTGTAGCAGGAGTAGT | |
| CATCAAAGCCAAAAGCTGGAAACCACCTGAGTGTCTACCAGCAGATGATTGGAATAACCAA | |
| TGGTAAATCAATATCTAAAACTTAACTATTCAGATAATAAGGTCTCATATAGTCCAGATTG | |
| GCCTAGAGCATCCCTCCTTTGATCTTCCCAAAGAGGGGATTCAGGAGGGAGAGTGTGACTG | |
| GGCANAGAGGAGGGAGGGCCTATGAACAAGATGTAAATTGAATTAATTAAAAAAAAAGATG | |
| ACCTTGACTTCTGCTACTCCTGCCCCTACCTACTGAGTGTTGGAATTACAGGCACAACCT | |
| CATCATGCGCATTCTTTAGTGCTGGTGATCAAACCCAGGGCTTCATGCATCCTAGCTAAGC | |
| ACTCTACCAACTCAGCTATTTTTCAGCCCTAGCAATGTNCTTCTGAATGGCTCATGGGTCC | |
| AAGAGGAACTCAACAGAGAAATTATAGAATAGTTTACTTCCATGGAAATGAAACCTCAGCA | |
| TCTCAGAATTGTGGGATATTGCTGAAGCAGAGGGAAGTTTCAAGCTTTGAGTGCTCCATTA | |
| GCAAAGAGGGAGGGACCAGACTGAGAGCTTCAGTGTTTATGTTATGAAAATAGAAAAAGCT | |
| GAATGCATTACACCNAGAGCAAACTAGGAAGGAACTAATGAAGAATAAAAATTACTGAAAT | |
| TATTAGAAAACAAAAACAATAAAATTAACCAAAAGCTAATTCTTTGAAAATATATTAAATT | |
| GTCCCTTTGGCCCAATTGATAAAAAAAAAAA. | |

### EXAMPLE 17

### AGT-116 Gene Expression

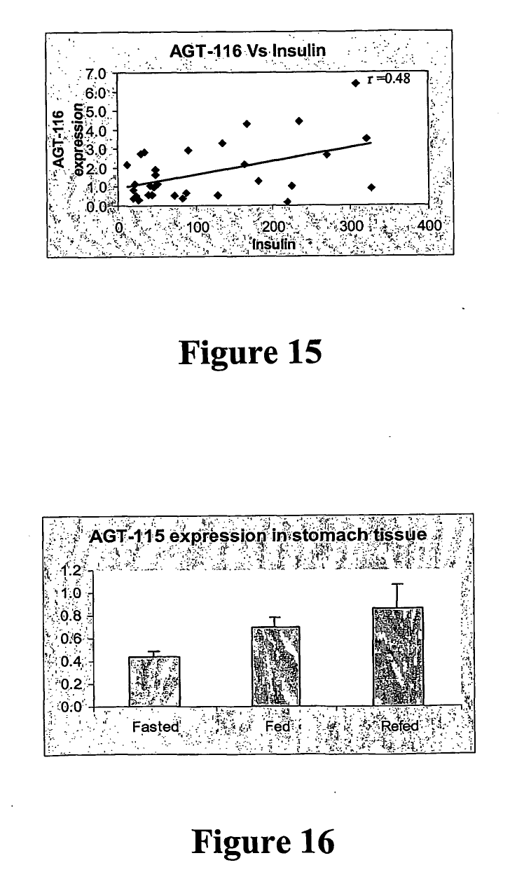

SYBR green RT-PCR on stomach cDNA from fasted, fed and re-fed animals showed that gene expression was not significantly different between groups although there was a trend for expression to be increased in fed animals (n≧13 in each group, FIG. 14). AGT-116 expression was positively correlated to plasma insulin concentration (p=0.007, FIG. 15), but was not correlated with body weight, stomach weight (content removed), stomach content or plasma glucose.

### EXAMPLE 18

### Partial Sequence of Psammomys Obesus AGT-115

AGT-115 was identified by differential display of fasted, fed and re-fed stomach cDNA from Psammomys obesus.

The partial nucleotide sequence is as follows:—

| | |
|---|---|
| TTCTAGATAGCCTNACAGCTTTGCTCTCATATTGTATTTAATTGCTGATACAGTATNTCC | [SEQ ID NO: 7] |
| TTGGAGGTCTTTTCTCTGTAATCTACACCTCTAGAATTGTTTCTGGCCTCTGCCCATTTC | |
| TGTTAACACACAGAACTCTTTGGGTTACCACTGCACAAAATTGCTTATTTAGGCCAGGAA | |
| ATGTCATGAATGTCTTCCATCTCANCATTATAGAGGCCTAGGAGGCAGAAGAAAAAGACC | |
| AAGTTTGGACAAGACAAGGCTATATAAAACTGGCCTCAAAAATAAATAAAATTTCTTATC | |
| TGTGAAAAAAAAAAA. | |

### EXAMPLE 19

### AGT-115 Gene Expression

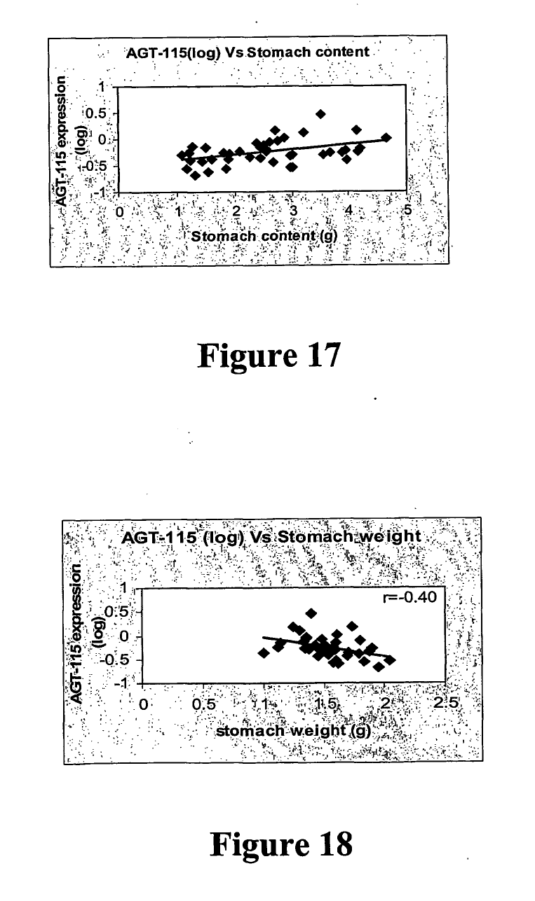

SYBR green RT-PCR on stomach cDNA from fasted, fed and re-fed animals showed that gene expression was significantly higher in fed (p=0.019) and re-fed (p=0.01) when compared to fasted animals (n≧13 in each group, FIG. 16). AGT-115 (log) expression was positively correlated with stomach content (p=0.011, FIG. 17) and negatively correlated to stomach weight (content removed) (p=0.011, FIG. 18), but was not correlated with body weight, plasma glucose or insulin.

### EXAMPLE 20

### AGT-115 Gene Homology

Blast searches have not identified any homology with any EST's or known genes. AGT-115 is currently undergoing RACE PCR to obtain more sequence.

### EXAMPLE 21

### Partial Sequence of Psammomys Obesus AGT-108

AGT-108 was identified by differential display of fasted, fed and re-fed stomach cDNA from Psammomys obesus.

The partial nucleotide sequence is as follows:—

| | |
|---|---|
| AAAACCTGGATGTAATAAATAAGATCATGGAAAGCTTTATGTGAAGAAAATTGAATGTTA | [SEQ ID NO: 8] |
| TAGTATAAAAAAGATATTTATGTATGTNCAGTTTGCTAAAGCCAAGTTTTGTTTGTTGAT | |
| TTCTTTGCATTTATTATAGATTCTATAAGTAAAAAAAAAAA. | |

### EXAMPLE 22

### AGT-108 Gene Expression

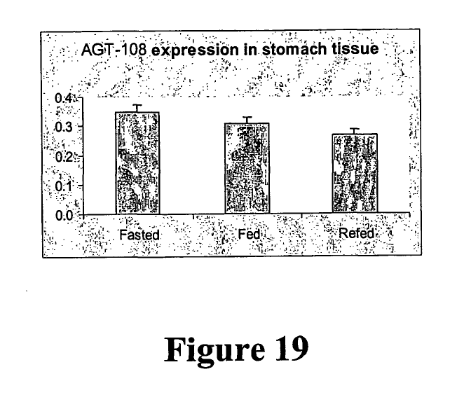

SYBR green RT-PCR on stomach cDNA from fasted, fed and re-fed animals showed that AGT-108 expression was significantly higher in fasted compared to re-fed animals (p=0.022) but not compared to fed animals (n≧13 in each group, FIG. 19). AGT-108 expression was not significantly correlated with stomach weight, stomach content, bodyweight, post-glucose or post-insulin.

### EXAMPLE 23

### AGT-108 Homology

Blast searches with the partial sequence for AGT-108 revealed 92% homology with human DNA sequence from clone RP1-130E4 on chromosome 6q24.2-25.3. Contains the 3′ end of the ESR1 gene for estrogen receptor 1, the 3′ end of the gene KIAA0796.

Searches with KIAA0796, a human cDNA found as part of a project to identify novel genes expressed in the brain, revealed high homology (82%) with Syne-1B and is believed to be the human ortholog.

Synaptic nuclear envelope Syne-1 was identified using the yeast 2 hybrid system and was found to bind cytoplasm C domain of MuSK (Apel et al., J. Biol. Chem. 275: 31986-31995, 2000). MuSK is a critical component of the receptor for agrin, a nerve-derived proteoglycan signal critical for all aspects of post-synaptic differentiation (including transcriptional specialization of synaptic nuclei in muscle cells, crucial for the development of functional neuromuscular junctions). Syne-1B, an isoform of Syne-1, is associated with nuclear envelopes in skeletal, cardiac and smooth muscle cells (Apel et al., 2000, supra). Syne-1 isoforms are selectively associated with synaptic nuclei (Apel et al., 2000, supra). It has been suggested that due to its localization and structure, it may be involved in the formation or maintenance of nuclear aggregates at the neuromuscular junction.

Syne-1B expression has been shown in adult human brain, heart, kidney, liver, lung, pancreas, skeletal muscle, but not in the placenta (Apel et al., 2000, supra). In human foetal tissue, expression has been confirmed in brain, heart, kidney, liver, lung, skeletal muscle, spleen, but not in thymus (Apel et al., 2000, supra). Syne-1 has been shown to be associated with nuclear envelopes in muscle cells and associated with intramuscular arterioles but not in venules (Apel et al., 2000, supra). Syne-1 appears to be more predominant in myotubes than in myoblasts (Apel et al., 2000, supra). Although present in the myonuclei of skeletal muscle fibers, levels are highest at the post-synaptic membrane (Apel et al., 2000, supra).

Syne-1 A and B have common 3′ sequences but distinct 5′ sequences, which is thought to arise through alternative splicing or from separate promoters to give a ˜4.7 kb and ˜10 kb sequence respectively. Syne-1B has ˜10 kb of specific sequence plus 919 aa of common seq to Syne-1A (Apel et al., 2000, supra). Syne-1B sequence contains 15 “spectrin repeats”, these 100-aa long domains were first described in the cytoskeletal protein spectrin, and have also been found in many rod-shaped proteins that are components of or associated with the cytoskeleton. The human DNA sequence is located on chromo. 6q 24.2-25.3 (Nagase et al., DNA Res. 5: 277-286, 1998).

Because SYNE-1B is associated with the cytoskeleton and the neuromuscular junction, it may be directly affected by the degree of stretch of the stomach wall and could communicate with appetite centres in the CNS via the neuromuscular junction. Blocking the production or actions of SYNE-1 B in the stomach could suppress appetite.

### EXAMPLE 24

### Gene Expression Studies

Gene expression in each cDNA sample was quantitated using Taqman PCR technology on an ABI Prism 7700 sequence detector (PE Applied Biosystems, Foster City, Calif.). β-actin was used as an endogenous control to standardize the amount of cDNA added to a reaction. PCR conditions were 50° C. for 2 min, 95° C. for 10 min followed by 40 cycles of 95° C. for 15 sec and 60° C. for 1 min. All samples were assayed in duplicate. For β-actin, a fluorogenic probe which had the reporter dye FAM attached to the 5′-end and the quencher dye TAMRA attached to the 3′-end was used with Taqman Universal PCR master Mix (Applied Biosystems). For all other genes examined no probe was used and SYBR Green Master Mix (Applied Biosystems) was used instead. The level of expression of β-actin, the “house-keeping gene” was examined in each group and shown not to be altered under any of the experimental conditions examined.

Primer and probe sequences were as follows:

| | | |
|---|---|---|
| β-actin (ISR) | | |
| Forward: | 5′-GCAAAGACCTGTATGCCAACAC-3′ | [SEQ ID NO: 9] |
| Reverse: | 5′-GCCAGAGCAGTGATCTCTTTCTG-3′ | [SEQ ID NO: 10] |
| Probe: | 5′Fam-TCCGGTCCACAATGCCTGGGAACAT-Tamra3′ | [SEQ ID NO: 11] |
| AGT-107 | | |
| Forward: | 5′-GCAGCTAGACATCTCAGAGGAAGA-3′ | [SEQ ID NO: 12] |
| Reverse: | 5′-GGAAAGGCACAGTGGAGTTTG-3′ | [SEQ ID NO: 13] |
| AGT-114 | | |
| Forward: | 5′-CACCAGTGGTGGCAGCAA-3′ | [SEQ ID NO: 14] |
| Reverse: | 5′-CAGCAGCCACTTCCGATTTC-3′ | [SEQ ID NO: 15] |
| AGT-116 | | |
| Forward: | 5′-TGAAAATAGAAAAAGCTGAATGC-3′ | [SEQ ID NO: 16] |
| Reverse: | 5′-TTCTTCATTAGTTCCTTCCTAGC-3′ | [SEQ ID NO: 17] |
| AGT-115 | | |
| Forward: | 5′-GGCCTCTGCCCATTTCTGT-3′ | [SEQ ID NO: 18] |
| Reverse: | 5′-ATTCATGACATTTCCTGGCCTAA-3′ | [SEQ ID NO: 19] |
| AGT-108 | | |
| Forward: | 5′-TGGATGTAATAAATAAGATCATGGAAAGC-3′ | [SEQ ID NO: 20] |
| Reverse: | 5′-AAGAAATCAACAAACAAAACTTGGC-3′ | [SEQ ID NO: 21] |
| AGT-117 | | |
| Forward: | 5′-TGGTGCTGAGGGTGATTTGA-3′ | [SEQ ID NO: 22] |
| Reverse: | 5′-TATCACAACAGTCCCTAGGTCTCATA-3′ | [SEQ ID NO: 23] |
| AGT-110 | | |
| Forward: | 5′-GAGCGCCGAGCTCTCAAC-3′ | [SEQ ID NO: 24] |
| Reverse: | 5′-CTGAGGCGGGACAGGAATAA-3′ | [SEQ ID NO: 25] |
| AGT-199 | | |
| Forward: | 5′-ACATGTCTGTTTTGGTGGCAATA-3′ | [SEQ ID NO: 26] |
| Reverse: | 5′-TCAAAGGACACTACAGAACCACTGTT-3′ | [SEQ ID NO: 27] |

### EXAMPLE 25

### Statistical Analysis

All data are expressed as mean+/−S.E.M. A one-way analysis of variance in combination with either the Least Significant Difference or Games-Howell post hoc test were used to compare means between more than two groups, and T-tests were used where appropriate. A two-tailed Pearson correlation was performed to analyse relationships between gene expression and phenotypic measures and all variables were tested for normality before use. Where variables were not normally distributed they were log transformed to approximate a normal distribution. A significance value of p<0.05 was used in all cases.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

1-58. Canceled.

59. An isolated nucleic acid molecule comprising a sequence of nucleotides encoding, or complementary to a sequence encoding, a protein or derivative or homolog thereof, wherein said nucleic acid molecule is differentially expressed in at least one of liver or stomach tissue of either (a) obese animals compared to lean animals or (b) fed animals compared to fasted animals, wherein the nucleic acid molecule is selected from:
(i) a nucleic acid molecule comprising a nucleotide sequence selected from the group consisting of SEQ ID NOS:1-8, 28, 32 (human AGT-114), 33, and 35;
(ii) a nucleic acid molecule comprising a nucleotide sequence at least about 70% identical to a nucleotide sequence selected from the group consisting of SEQ ID NOS:1-8, 28, 32 (a human AGT-114), 33, and 35; and
(iii) a nucleic acid molecule capable of hybridizing to a nucleotide sequence selected from the group consisting of SEQ ID NOS:1-8, 28, 32 (human AGT-114), 33, and 35 or its complementary form under low stringency conditions.

60. The isolated nucleic acid molecule of claim 59 wherein the nucleic acid molecule comprises the nucleotide sequence set forth in SEQ ID NO: 1.

61. The isolated nucleic acid molecule of claim 59 wherein the nucleic acid molecule comprises the nucleotide sequence set forth in SEQ ID NO:2.

62. The isolated nucleic acid molecule of claim 59 wherein the nucleic acid molecule comprises the nucleotide sequence set forth in SEQ ID NO:3.

63. The isolated nucleic acid molecule of claim 59 wherein the nucleic acid molecule comprises the nucleotide sequence set forth in SEQ ID NO:4.

64. The isolated nucleic acid molecule of claim 59 wherein the nucleic acid molecule comprises the nucleotide sequence set forth in SEQ ID NO:5.

65. The isolated nucleic acid molecule of claim 59 wherein the nucleic acid molecule comprises the nucleotide sequence set forth in SEQ ID NO:6.

66. The isolated nucleic acid molecule of claim 59 wherein the nucleic acid molecule comprises the nucleotide sequence set forth in SEQ ID NO:7.

67. The isolated nucleic acid molecule of claim 59 wherein the nucleic acid molecule comprises the nucleotide sequence set forth in SEQ ID NO:8.

68. The isolated nucleic acid molecule of claim 59 wherein the nucleic acid molecule comprises the nucleotide sequence set forth in SEQ ID NO:28.

69. The isolated nucleic acid molecule of claim 59 wherein the nucleic acid molecule comprises the nucleotide sequence set forth in SEQ ID NO:32 (human AGT-114).

70. The isolated nucleic acid molecule of claim 59 wherein the nucleic acid molecule comprises the nucleotide sequence set forth in SEQ ID NO:33.

71. The isolated nucleic acid molecule of claim 59 wherein the nucleic acid molecule comprises the nucleotide sequence set forth in SEQ ID NO:35.

72. An isolated protein molecule encoded by the nucleic acid molecule according to any one of claims 59-71.

73. The isolated nucleic acid molecule according to any one claims 59-71 wherein the nucleic acid molecule is RNA.

74. An isolated protein encoded by a nucleotide sequence selected from the group consisting of SEQ ID NOS:1-8, 28, 32 (human AGT-114), 33, and 35.

75. An isolated protein encoded by a nucleic acid molecule that is differentially expressed in at least one of stomach tissue or liver tissue of either (a) obese animals compared to lean animals or (b) fed animals compared to fasted animals, wherein the protein is selected from the group consisting of:
(i) a protein encoded by a nucleotide sequence selected from the group consisting of SEQ ID NOS:1-8, 28, 32 (human AGT-114), 33, and 35, or a derivative, homolog or analog of such a nucleotide sequence, or a derivative, homolog, analog, chemical equivalent or mimetic of said protein;
(ii) a protein comprising an amino acid sequence at least about 70% identical to an amino acid sequence encoded by a nucleic acid molecule comprising a nucleotide sequence selected from the group consisting of SEQ ID NOS:1-8, 28, 32 (human AGT-114), 33, and 35, or a derivative, homolog, analog, chemical equivalent, or mimetic of said protein; and
(iii) a protein encoded by a nucleic acid molecule capable of hybridizing to a nucleotide sequence selected from the group consisting of SEQ ID NOS:1-8, 28, 32 (human AGT-114), 33, and 35, or a derivative, homolog, or analog thereof, under low stringency conditions.

76. A method for modulating expression of at least one nucleic acid molecule selected from the group consisting of AGT-117, AGT-110, AGT-119, AGT-107, AGT-114, AGT-116, AGT-115 and AGT-108 in a mammal, said method comprising contacting at least one nucleic acid molecule selected from the group consisting of AGT-117, AGT-110, AGT-119, AGT-107 AGT-114, AGT-116, AGT-115, and AGT-108 with an effective amount of a modulator of at least one nucleic acid molecule selected from the group consisting of AGT-117, AGT-110, AGT-119, AGT-107, AGT-114, AGT-116, AGT-115, and AGT-108 expression for a time and under conditions sufficient to up-regulate or down-regulate or otherwise modulate expression of at least one nucleic acid molecule selected from the group consisting of AGT-117, AGT-110, AGT-119, AGT-107, AGT-114, AGT-116, AGT-115, and AGT-108.

77. A method of modulating activity of at least one protein selected from the group consisting of AGT-117, AGT-110, AGT-119, AGT-107, AGT-114, AGT-116, AGT-115, and AGT-108 in a mammal, said method comprising administering to said mammal a modulating effective amount of a molecule for a time and under conditions sufficient to increase or decrease an activity of at least one protein selected from the group consisting of AGT-117, AGT-110, AGT-119, AGT-107, AGT-114, AGT-116, AGT-115, and AGT-108.

78. A method of treating a mammal suffering from a condition characterized by at least one symptom of a condition selected from the group consisting of obesity, anorexia, diabetes and energy imbalance, said method comprising administering to said mammal an effective amount of an agent for a time and under conditions sufficient to modulate the expression of at least one nucleic acid molecule selected from the group consisting of AGT-117, AGT-110, AGT-119, AGT-107, AGT-114, AGT-116, AGT-115 and AGT-108, or sufficient to modulate an activity of at least one protein selected from the group consisting of AGT-117, AGT-110, AGT-119, AGT-107, AGT-114, AGT-116, AGT-115 and AGT-108.

79. A method of treating a mammal suffering from a disease condition characterized by at least one symptom of a condition selected from the group consisting of obesity, anorexia, diabetes and energy imbalance, said method comprising administering to said mammal an effective amount of either at least one protein selected from the group consisting of AGT-117, AGT-110, ACT-119, AGT-107, AGT-114, AGT-116, AGT-115 and AGT-108, or at least one nucleic acid molecule selected from the group consisting of AGT-117, AGT-110, AGT-119, AGT-107, AGT-114, AGT-116, AGT-115 and AGT-108.

80. A method for treating a condition selected from the group consisting of obesity, anorexia, diabetes and energy imbalance comprising administering to a mammal in need thereof an agent capable of modulating the expression of at least one nucleic acid molecule selected from the group consisting of AGT-117, AGT-110, AGT-119, AGT-107, AGT-114, AGT-116, AGT-115 and AGT-108, or a derivative, homolog, or analog thereof.

81. A method for treating a condition selected from the group consisting of obesity, anorexia, diabetes and energy imbalance comprising administering to a mammal in need thereof an agent capable of modulating an activity of at least one protein selected from the group consisting of AGT-117, AGT-110, AGT-119, AGT-107, AGT-114, AGT-116, AGT-115 and AGT-108, or a derivative, homolog, analog, chemical equivalent, or mimetic thereof.

82. A method for treating a condition selected from the group consisting of obesity, anorexia, diabetes and energy imbalance comprising administering at least one nucleic acid molecule selected from the group consisting of AGT-117, AGT-110, AGT-119, AGT-107, AGT-114, AGT-116, AGT-115 and AGT-108 or a derivative, homolog, or analog thereof.

83. A method for treating a condition selected from the group consisting of obesity, anorexia, diabetes and energy imbalance comprising administering at least one protein selected from the group consisting of AGT-117, AGT-110, AGT-119, AGT-107, AGT-114, AGT-116, AGT-115 and AGT-108, or a derivative, homolog, analog, chemical equivalent, or mimetic thereof.

84. A composition comprising a modulator of expression of at least one nucleic acid molecule selected from the group consisting of AGT-117, AGT-110, AGT-119, AGT-107, AGT-114, AGT-116, AGT-115 and AGT-108 and at least one pharmaceutically acceptable carrier or diluent.

85. A composition comprising a modulator of an activity of at least one protein selected from the group consisting of AGT-117, AGT-110, AGT-119, AGT-107, AGT-114, AGT-116, AGT-115, and AGT-108 and at least one pharmaceutically acceptable carrier or diluent.

86. A method for detecting a protein selected from the group consisting of AGT-117, AGT-110, AGT-119, AGT-107, AGT-114, AGT-116, AGT-115 and AGT-108, or a derivative or homolog thereof, in a biological sample from a subject, said method comprising contacting said biological sample with an antibody specific for a protein selected from the group consisting of AGT-117, AGT-110, AGT-119, AGT-107, AGT-114, AGT-116, AGT-115 and AGT-108, or an antigenic derivative or homolog thereof, for a time and under conditions sufficient for a complex to form, and then detecting said complex.
